(19) United States
(12) Patent Application Publication
(10) Pub. No.: US 2021/0236050 A1
(43) Pub. Date: Aug. 5, 2021

(54) DYNAMIC ANATOMIC DATA COLLECTION AND MODELING DURING SLEEP

(71) Applicant: Jerry Chi Hu, Soldotna, AK (US)

(72) Inventor: Jerry Chi Hu, Soldotna, AK (US)

(21) Appl. No.: 16/899,209

(22) Filed: Jun. 11, 2020

Related U.S. Application Data
(60) Provisional application No. 62/969,974, filed on Feb. 4, 2020.

(51) Int. Cl. A61B 5/00; G16H 10/60; G16H 40/67; G16H 50/20; G16H 50/30; G16H 15/00; G16H 50/50; A61B 7/00; A61C 19/05; A61B 5/0205; A61B 5/16; A61B 5/0488; A61B 5/0476; A61B 5/1455

(52) U.S. Cl. CPC A61B 5/4812; A61B 5/0816; G16H 40/67; G16H 50/20; G16H 50/30; G16H 15/00; G16H 50/50; A61B 7/003; A61B 5/742; A61C 19/05; A61B 5/4542; A61B 5/0205; A61B 5/163; A61B 5/0488; A61B 5/0476; A61B 5/4818; A61B 5/4557; A61B 5/14551; A61B 5/0088; G16H 10/60

(57) ABSTRACT

A system, method and appliance provides sleep-related disorder data collection, assessment, and visual representation. The appliance may be configured to be worn during a sleep cycle of an individual and may include or be operatively connected to various sensors that collect physiological data of the individual during the sleep cycle. Aspects of the system are configured to correlate data obtained from various sensors/data sources during an individual's sleep cycle, determine relevant events associated with sleep-related disorders based on the obtained data, provide summaries and information about the determined relevant events, and generate and provide dynamic visualizations of the individual's anatomy corresponding to the determined relevant events. A dynamic visualization may include a 3D model of the individual's airway and mandibular anatomy that, when played back, morphs to represent dynamics of the individual's anatomy during the relevant event.

### CROSS REFERENCE TO RELATED APPLICATIONS

This application claims the benefit of U.S. Provisional Application No. 62/969,974, having the title of “Dynamic Anatomic Data Collection and Modeling During Sleep” and the filing date of Feb. 4, 2020, which is incorporated herein by reference in its entirety.

### BACKGROUND

With the prevalence of sleep disorders amongst the general population, the detection of sleep-related disorders is important for enabling individuals, with the help of a healthcare professional, to treat their disorder. For example, sleep disorders, such as apneas, hypo-apneas, mixed apneas, respiratory effort-related arousals (RERAs), sleep bruxism, obstructed airway-associated head/neck movement disorders, temporomandibular disorders (TMD), etc., if untreated, can increase the risk of various health problems, such as obesity, diabetes, cardiovascular disease, and depression. The collapsibility of an individual's airway can have a correlation to the severity of certain sleep-related disorders. Accordingly, evaluation of an individual's airway can enable an identification of the person's anatomic risk factors, for determining a therapy for treating or managing an identified problem, and/or for determining the efficacy of a treatment.

Current methods of detecting sleep-related disorders may rely on acoustic reflection airway assessments, cone beam computed tomography scans, X-rays, Lateral Cephalometrics, ultrasounds, etc. However, current technologies do not provide for various physiological data of the individual to be collected during a sleep cycle or full sleep period (e.g., throughout a night) that can be correlated and used to provide measurements of the individual's airway during sleep and to generate dynamic 3-dimensional (3D) visualizations of the individual's airway and mandible corresponding to a determined relevant event. For example, because of the nature of the equipment required to perform these assessments, they cannot practically be used while the individual is asleep or in a non-clinical setting. As can be appreciated, assessments made during an individual's awake cycle may not provide an accurate evaluation of movements and measurements of the individual's airway during sleep. Moreover, the assessment results are limited to 2-dimensional (2D) visualizations, such as 2D line graphs. While changes in the individual's airway may be identified by observing changes amongst a plurality of 2D line graphs, this does not provide an anatomically-correct visualization that can transform to represent movement, size, or shape of dynamic anatomy, such as the human airway and mandible associated with a relevant event during a sleep cycle. Another current technology may include a cone beam computed tomography (CBCT) image. However, current CBCT technologies are limited to providing a static capture of a region of interest at a moment in time, which does not provide a way to visualize movement, size, or shape of the human airway and mandible in association with a relevant event during a sleep cycle.

Accordingly, a method and system are needed for improved data collection during a sleep cycle and improved anatomical visualization, communication, research, and metrics. It is with respect to these and other general considerations that embodiments have been described. Also, while relatively specific problems have been discussed, it should be understood that the embodiments should not be limited to solving the specific problems identified in the background.

### SUMMARY

The disclosure generally relates to a system and method for providing sleep-related disorder data collection, assessment, and visual representation. Aspects of a Dynamic Recorded Events of Associated Movements in Sleep (DREAMS) system described herein are configured to record physiological parameter data of an individual during one or more sleep cycles, evaluate the collected data, and generate results including information regarding data received and/or determined by the system for use in a variety of clinical and/or educational applications. The physiological parameter data may include data continually collected from various data sources while the individual is asleep, and may include data such as acoustical measurement data of at least a portion of the individual's airway, mandibular movement data, bite force data, as well as other physiological data (e.g., home sleep test data, polysomnogram data) associated with determinants of various sleep-related disorders (e.g., breathing disorders, movement disorders, other disorders). The system may be configured to analyze the collected data as part of determining relevant readings and events that may be indicative of a sleep-related disorder, correlate identified events with other physiological data related to the events (e.g., based on time), and generate output/results based on data received or determined by the system, such as graphs, dynamic 3D visualizations, measurements, summarized information about the collected data and about the identified events, etc. For example, the output/results may be provided to a user (e.g., the individual, a healthcare professional) via audible output via a speaker and/or visual output displayed on a screen.

The dynamic 3D visual visualizations may include a computer aided design (CAD) model that is generated based on an integration of static 3D image data, such as a set of cone beam computerized tomography (CBCT) images, and baseline data obtained from one or more of an acoustic reflection measurement device, a positioning/movement sensor, and a pressure sensor. The 3D model may be transformed to represent anatomic dynamics associated with the individual's airway, mandible positions/movements, and/or occlusal forces corresponding to a relevant event during the individual's sleep that may be indicative of or associated with a sleep-related disorder. The dynamic 3D visual representation provides an anatomically-correct representation of the patient's anatomy (e.g., in comparison with a 2D representation), and can enable a user to visually assess time-based changes to their airway and mandibular anatomy during a relevant event (e.g., jaw movements, clenching of teeth, airway collapses) occurring during a sleep cycle.

The collected data and dynamic 3D visual representation may provide the individual and the individual's healthcare provider with information on specific sites and measurements of airway obstruction, jaw movements, occlusal forces, and other related physiological data, such as blood-oxygen saturation levels, airflow, respiratory effort, heart rate, heart rhythm, breathing pattern, eye movement, muscle activity, brain activity, snoring and other noises made while sleeping, etc.

In a first aspect, a system for providing sleep-related disorder data collection, assessment, and visual representation is provided. In an example embodiment, the system includes at least one processor, a memory storage device including instructions that when executed by the at least one processor are configured to: receive physiological data of an individual during a sleep cycle, wherein the physiological data at least include acoustic measurement data of the individual's airway; analyze the physiological data for determining a relevant event associated with a sleep-related disorder; determine results associated with the relevant event; and provide output for display on a screen, wherein the output includes the results.

In another aspect, a method for providing sleep-related disorder data collection, assessment, and visual representation is provided. In an example embodiment, the method comprises: receiving physiological data of an individual during a sleep cycle, wherein the physiological data at least include acoustic measurement data of the individual's airway; analyzing the data for determining a relevant event associated with a sleep-related disorder; determining results associated with the relevant event; and providing output for display on a screen, wherein the output includes the results.

In another aspect, an apparatus for providing sleep-related disorder data collection, assessment, and visual representation is provided. In an example embodiment, the apparatus is an oral appliance device configured to be worn by an individual during a sleep cycle, the apparatus including or operatively connected to: an acoustic reflection measurement device configured to collect acoustic measurement data of the individual's airway; a positioning and movement sensor configured to collect positioning and movement data associated with mandibular movements; and a pressure sensor configured to collect pressure data associated with occlusal forces.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

Non-limiting and non-exhaustive examples are described with reference to the following figures:

FIG. 1 is a block diagram of an example environment in which a system of the present disclosure can be implemented;

FIGS. 2A-E are illustrations of example embodiments of an oral appliance device that may be used to collect physiological data during a sleep cycle;

FIGS. 3A-F are illustrations of example embodiments of an acoustic reflection measurement device;

FIGS. 4A-B are illustrations of example embodiments of a positioning/movement sensor;

FIGS. 5A-G are illustrations of example embodiments of a pressure sensor;

FIG. 6 is a block diagram showing components of an example system of the present disclosure;

FIGS. 7A-F are illustrations of example results provided by the system;

FIG. 8 is a flow diagram depicting general stages of an example process for providing sleep-related disorder data collection, assessment, and visual representation according to an embodiment;

FIG. 9 is a flow diagram depicting general stages of an example process for generating a 3D visual representation representing anatomic dynamics associated with a relevant event during the sleep cycle; and

FIG. 10 is a block diagram illustrating example physical components of a computing device or system with which embodiments may be practiced.

### DETAILED DESCRIPTION

The following detailed description refers to the accompanying drawings. Wherever possible, the same reference numbers are used in the drawings and the following description to refer to the same or similar elements. While aspects of the present disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the present disclosure, but instead, the proper scope of the present disclosure is defined by the appended claims. Examples may take the form of a hardware implementation, or an entirely software implementation, or an implementation combining software and hardware aspects. The following detailed description is, therefore, not to be taken in a limiting sense.

The present disclosure provides a system, method, and apparatus for providing sleep-related disorder data collection, assessment, and visual representation. Aspects of the present disclosure are configured to record physiological parameter data of an individual during a sleep cycle, evaluate the collected data, and generate and provide results including information regarding data received and/or determined by the system for use in a variety of clinical and/or educational applications. The sleep cycle may include one or more sleep cycles. For example, a full sleep period (e.g., throughout a night) may be comprised of a plurality of sleep cycles. The physiological parameter data may include data continually collected from various data sources, and may include data such as acoustical measurement data of at least a portion of the individual's airway, mandibular movement data, bite force data, as well as other physiological data associated with determinants of various sleep-related disorders (e.g., breathing disorders, movement disorders, other disorders). The system may be configured to analyze the collected data as part of determining relevant readings and events that may be indicative of a sleep-related disorder, correlate identified relevant events with other data related to the events (e.g., based on time), and generate output/results based on data received and/or determined by the system, such as graphs, dynamic 3D visualizations, measurements, summarized information about the collected data and about the identified events, etc. Example clinical applications can include diagnosing an individual with a sleep-related disorder, determining efficacy of a treatment or therapy, verifying effectiveness of a rendered treatment or therapy, and other clinical applications.

Aspects of the present disclosure provide improvements to current diagnostic techniques by allowing data to be safely and non-intrusively collected while the individual is sleeping, thereby providing more accurate and relevant data for assessing sleep-related disorders. Additionally, aspects provide 3D visual representations of relevant events that provide a more anatomically-correct representation (e.g., in comparison to 2D representations) of the individual's anatomy (e.g., upper airway anatomy and mandible) and that can be dynamically viewed to enable the individual and healthcare providers to visually assess actual movements and dynamics of the anatomy. For example, the dynamic 3D visual representations may provide visually-observable information that can aid in the analysis and comprehension of the anatomy of the individual in relation to the assessment and/or treatment of sleep-related disorders. Although examples are given herein primarily involving assessment of an individual's airway, it will be recognized that the present disclosure is applicable to other types of lumens of an individual's body.

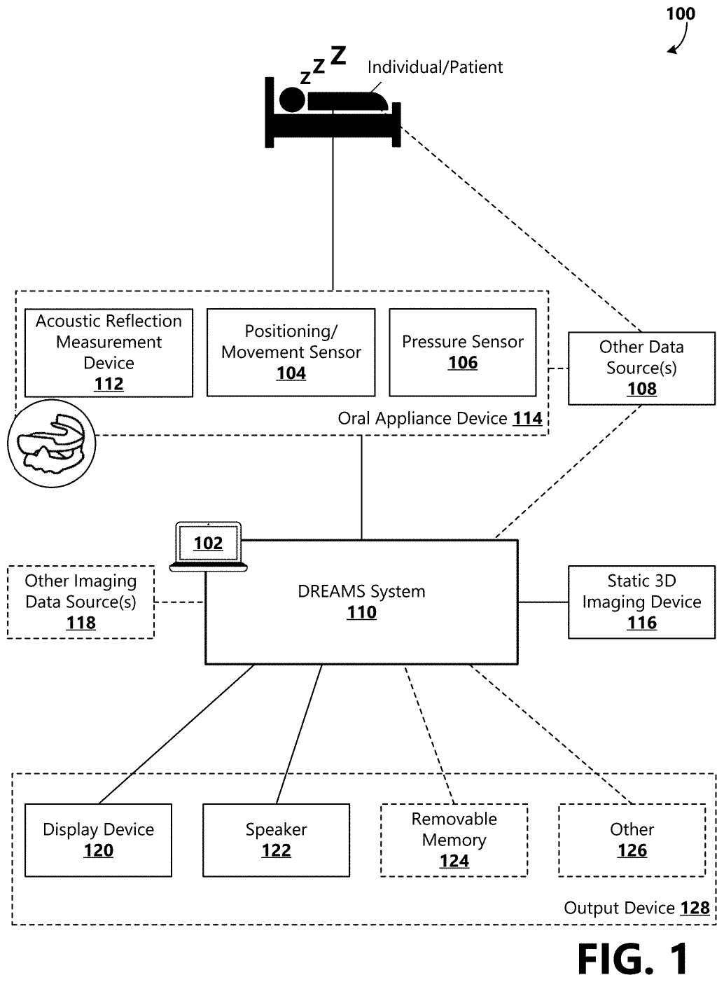

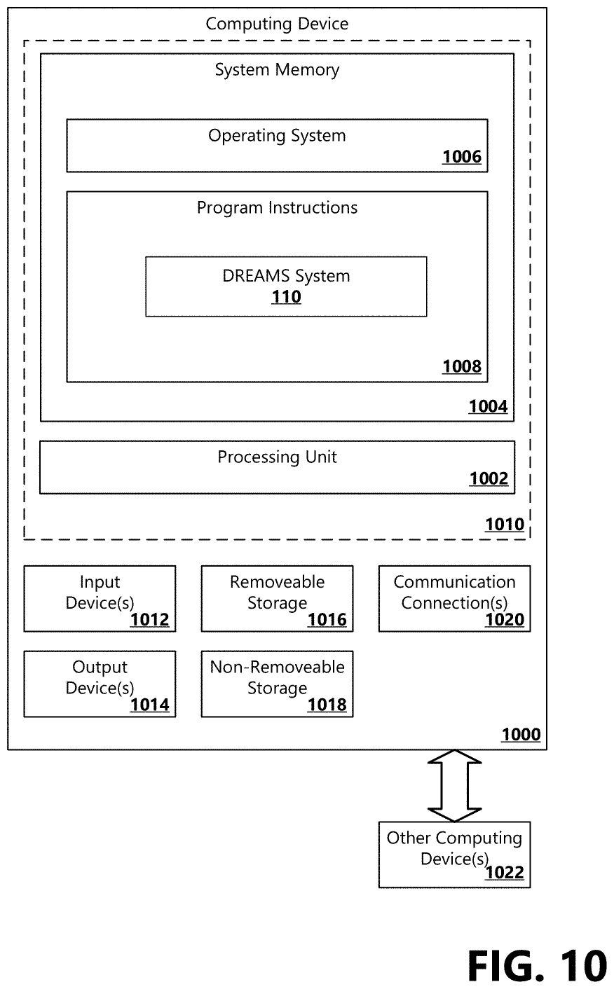

FIG. 1 is a block diagram of an example environment 100 in which a Dynamic Recorded Events of Associated Movements in Sleep (DREAMS) system 110 of the present disclosure can be implemented. As illustrated, the example environment 100 includes one or more computing devices 102. In one embodiment, the DREAMS system 110, sometimes hereinafter referred to as system 110, is illustrative of a computing device 102 or module that comprises at least one processor and a memory storage device including instructions that when executed by the at least one processor are configured to perform functionalities as described herein for providing sleep-related disorder data collection, assessment, and modeling. In another embodiment, the DREAMS system 110 is illustrative of a software application that can be executed by a computing device 102, which includes sufficient computer executable instructions that are operative or configured to perform functionalities as described herein for providing sleep-related disorder data collection, assessment, and modeling. For example, the DREAMS system 110 is operative or configured to perform functionalities associated with receiving physiological data of an individual while sleeping, evaluating the collected data for identifying determinants indicative of a sleep-related health disorder of the individual, and generating results associated with identified determinants for output. Applications may include thick client applications, which may be stored locally on the computing device 102, or may include thin client applications (i.e., web applications) that may reside on a remote server and be accessible over a network or a combination of networks (e.g., the Internet, wide area networks, local area networks). The computing device 102 may be one or more of various types of computing devices (e.g., a server device, a desktop computer, a tablet computing device, a mobile device or smartphone, a laptop computer, a laptop/tablet hybrid computing device, a large screen multi-touch display, or other type of computing device) configured to execute instructions for performing a variety of tasks. The hardware of these computing devices 102 is discussed in greater detail in regard to FIG. 10.

As mentioned above, the DREAMS system 110 is operative or configured to collect various physiological data of an individual while sleeping. According to an aspect, the physiological data are associated with physiological determinants indicative of and/or events associated with one or more sleep-related health conditions/disorders. Example sleep-related health conditions/disorders include, but are not limited to, apneas, hypo-apneas, mixed apneas, respiratory effort-related arousals (RERAs), sleep bruxism, obstructed airway-associated head/neck movement disorders, temporomandibular disorders (TMD), etc. According to an aspect, the physiological data are received from various data sources.

One example data source includes an acoustic reflection measurement device 112 operative or configured to obtain acoustic measurement data that represent a particular anatomic region of interest of the individual and to provide the acoustic measurement data and associated timing data to the DREAMS system 110. According to an embodiment, the acoustic reflection measurement device 112 is configured for intra-oral use during sleep for obtaining acoustic measurement data of the anatomic region of interest (e.g., oropharynx to hypopharynx/glottis of the airway) while the individual is sleeping. Acoustic reflection measurements may be taken while the user is in various positions (e.g., supine, prone, on left side, on right side). For example, acoustic reflection measurements taken while the user is in a supine position may be analyzed for identifying readings and events that may be indicative of positional sleep apnea (e.g., sleep-breathing difficulties associated with the supine position). The acoustic measurement data may represent one or more particular anatomic landmarks included in the anatomic region of interest, and the one or more anatomic landmarks may be automatically identified from the acoustic reflection measurements (e.g., cross-sectional area measurements, distance measurements from one or more reference points). The anatomic landmark identifications and measurements may be used as part of mapping an anatomic landmark between a baseline reading of the acoustic measurement data and the static 3D image data, and for identifying and tracking the anatomic landmark in additional acoustic measurement data received by the DREAMS system 110 while the individual is sleeping.

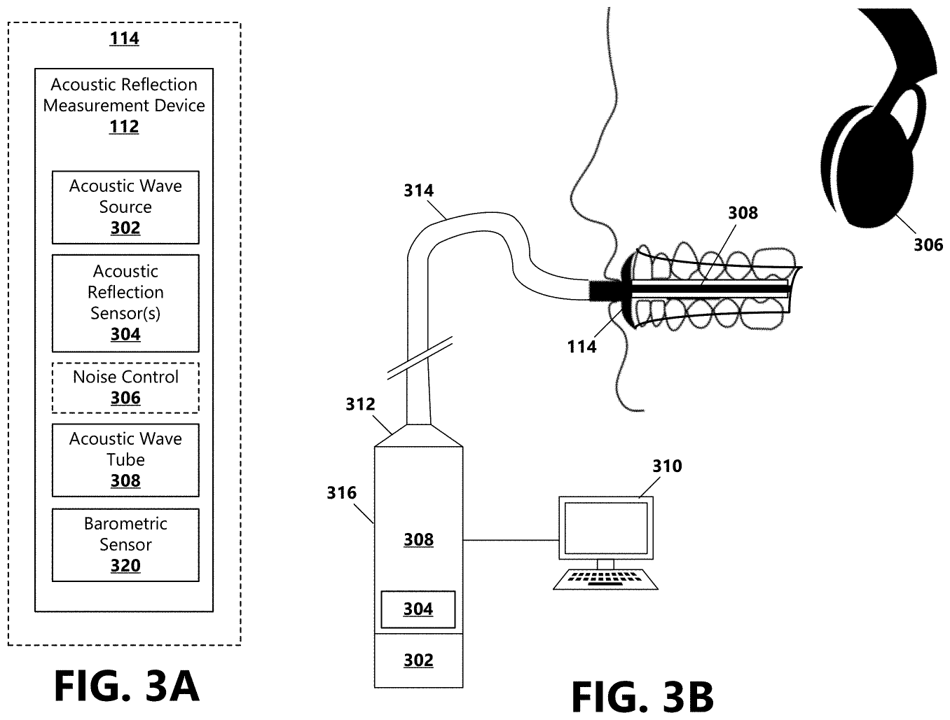

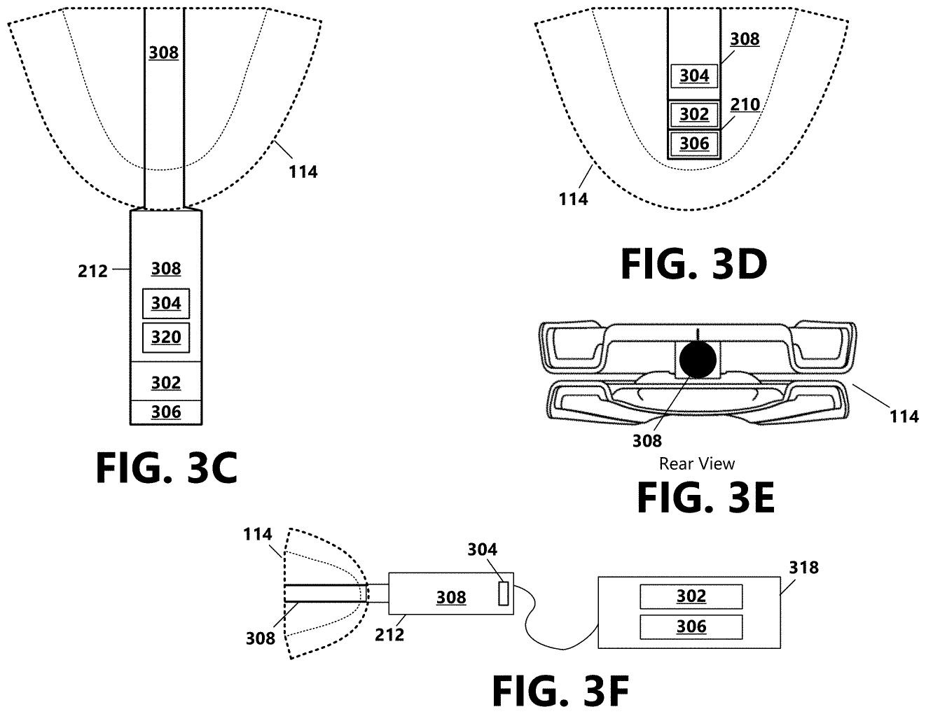

The acoustic reflection measurement data obtained and/or determined by the acoustic reflection measurement device 112 may be transmitted to the DREAMS system 110 and analyzed and correlated with other received data from one or more other data sources 108 and/or image data sources 118. The acoustic reflection measurement data may additionally be used to generate one or more 3D dynamic visualizations of the individual's airway. Example acoustic reflection measurement devices 112 are illustrated in FIGS. 3A-F and will be described further below.

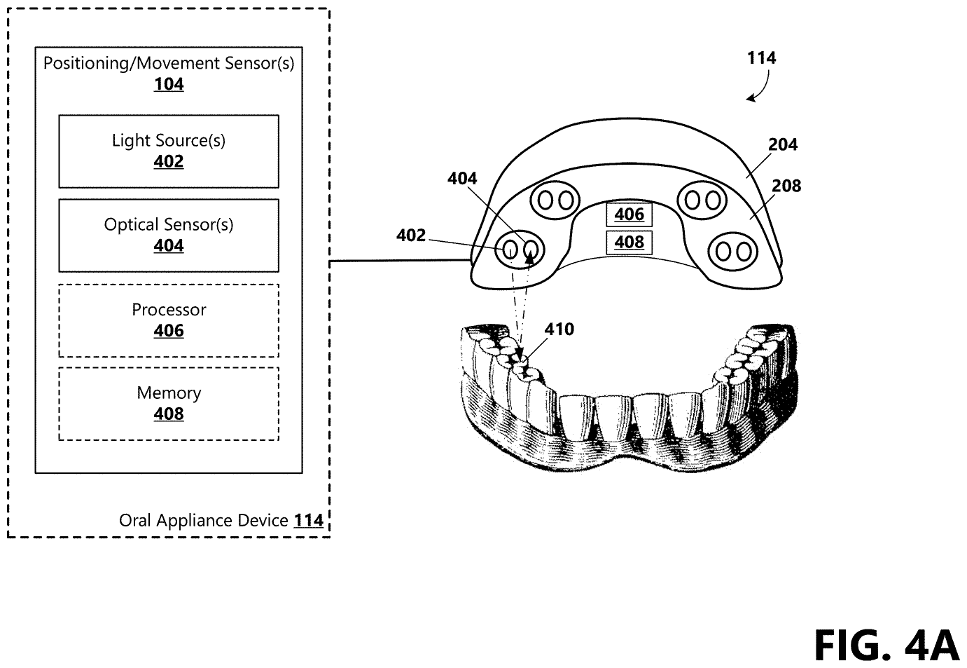

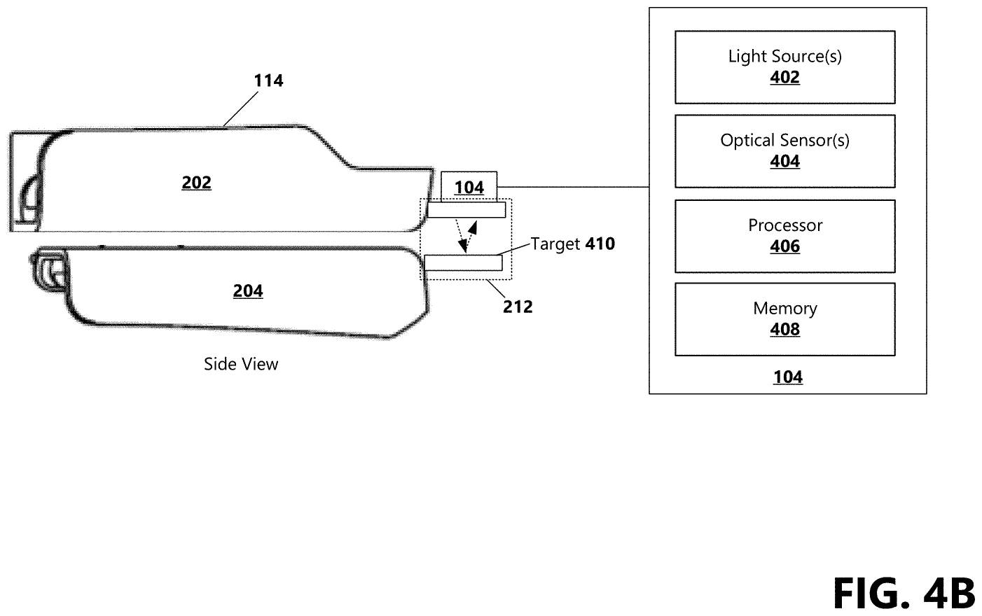

According to an embodiment, the oral appliance device 114 may include or be operatively connected to a positioning/movement sensor 104 configured as an additional data source to the DREAMS system 110. The positioning/movement sensor 104 is operative or configured to determine mandibular positions and movements (e.g., motion along three perpendicular axes (forwards or backwards, left or right, up or down), as well as rotation about three perpendicular axes (pitch, yaw and roll)), and to provide corresponding position and movement data and corresponding timing data to the DREAMS system 110. For example, the mandibular position and/or movements may be determined based on readings, obtained by the positioning/movement sensor 104, corresponding to positions and movements of one or more anatomic landmarks (e.g., teeth, mandible). Positions and/or movements of the individual's jaw/mandible may be associated with certain sleep sleep-related disorders. The position/movement data and associated timing data obtained and/or determined by the positioning/movement sensor 104 may be analyzed and correlated with other received data from one or more other data sources 108 and/or image data sources 118. The positioning/movement data may additionally be used to generate one or more 3D dynamic visualizations showing positioning and dynamics movements of at least the individual's mandible. Example positioning/movement sensors 104 included in or connected to an example oral appliance device 114 are illustrated in FIGS. 4A-B and will be described further below.

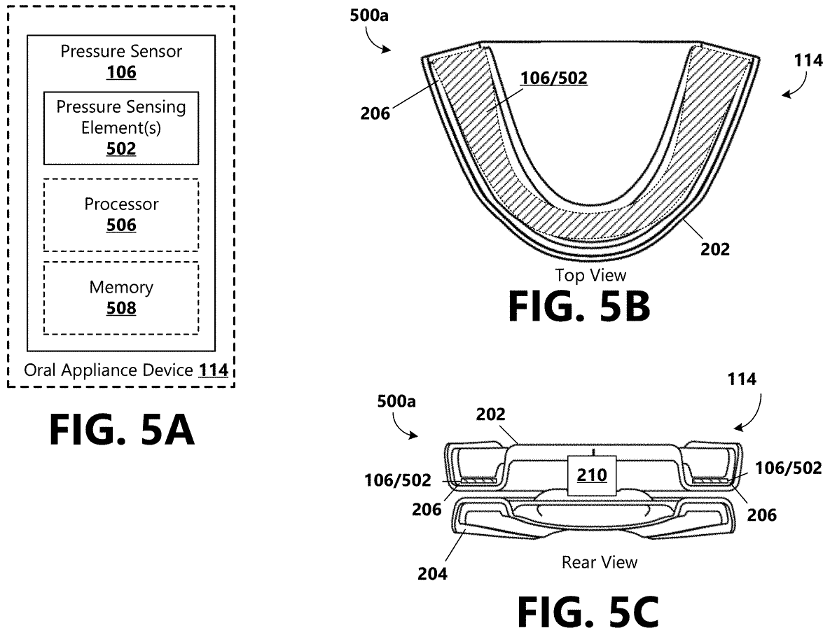

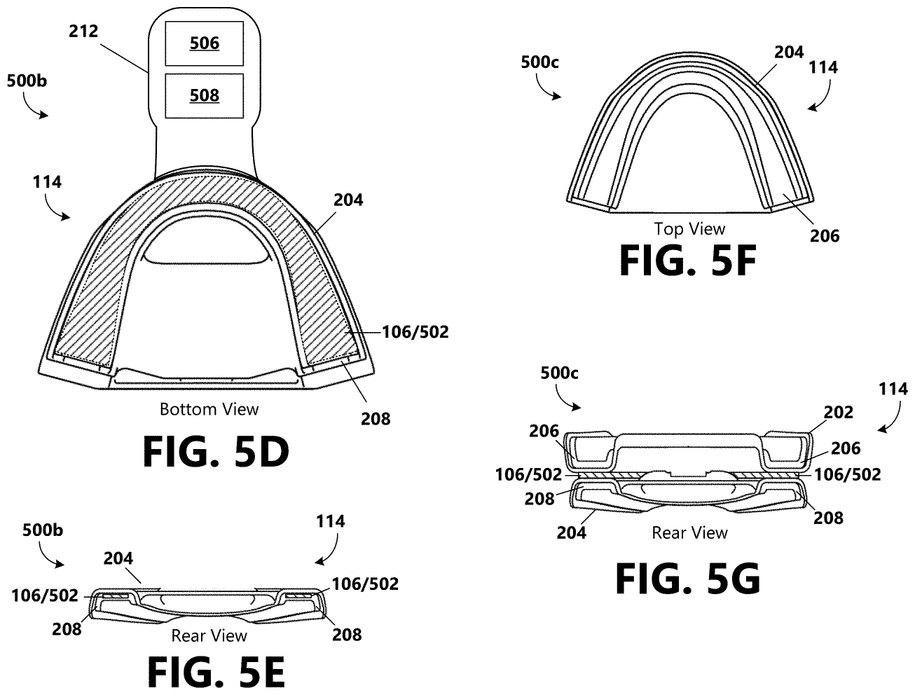

The oral appliance device 114 may further include or be operatively connected to a pressure sensor 106 that is configured as an additional data source to the DREAMS system 110. The pressure sensor 106 is operative or configured to continually obtain measurements associated with functional mandibular movement, the pressure/force of occlusion (i.e., force related to the relationship between the maxillary (upper) and mandibular (lower) teeth as they approach each other), and associated time data (e.g., time of contact, time of a pressure reading, duration of contact/pressure reading). Occlusion may include static occlusion (i.e., occurring when the jaw is closed or stationary) and dynamic occlusion (i.e., occurring when the jaw is moving). The pressure and timing data from pressure sensor 106 may be transmitted to the DREAMS system 110 and analyzed and correlated with other received data from one or more other data sources 108 and/or image data sources 118. The pressure data may be used to generate one or more 2D and/or 3D pressure visualizations showing locations of occlusal force, relative levels of occlusal force, and relative timing of occlusal force. In some examples, the pressure data may additionally be used, in combination with other received data, to generate one or more 3D dynamic visualizations. An example pressure sensor 106 included in or connected to an example oral appliance device 114 is illustrated in FIGS. 5A-G and will be described further below.

The DREAMS system 110 may be configured to receive data from one or more other data source(s) 108 that may be integrated with the oral appliance device 114 or which may comprise one or more separate devices. In some examples, one or more of the acoustic reflection measurement device 112, the positioning/movement sensor 104, and the pressure sensor 106 may be integrated with or operatively connected to the one or more other data source(s) 108. Examples of other data sources 108 may include a home sleep test unit, a polysomnogram unit, an autonomic nervous system and vascular assessment unit, or one or more other sensors configured to obtain physiological data of the individual during sleep. The home sleep test unit may be used by the individual in a home setting, while the polysomnogram unit may be used in a clinical setting. The home sleep test unit and polysomnogram unit may comprise various sensors, such as an electroencephalography (EEG) sensor (polysomnogram), a finger oxygen probe, a chest belt, a nasal tube, a microphone, and other sensors. The autonomic nervous system and vascular assessment unit may comprise various sensors, such as a blood pressure and arterial stiffness sensor, a photoplethysmographic (PPG) sensor, an ankle brachial index (ABI) sensor, a heartrate sensor, heartrate variability sensor, sudomotor function sensors, and other sensors. The one or more other data sources 108 may be utilized to obtain data related to the individual's blood-oxygen saturation levels, airflow, respiratory effort, heart rate, heartrate variability, heart rhythm, breathing pattern, eye movement, muscle activity, brain activity, snoring and other noises made while sleeping, blood pressure, arterial stiffness, sweat gland activity, etc.

The acoustic reflection measurement device 112, the positioning/movement sensor(s) 104, the pressure sensor(s) 106, and one or more of the other data sources 108 may be operatively connected to the DREAMS system 110 via wired, wireless, or a combination of wired and wireless connections. In example aspects, various wireless protocols can be used. In example embodiments, a WI-FI® protocol (802.11x) or a different wireless protocol (e.g., BLUETOOTH® including Bluetooth Low Energy, or BLE, cellular, RFID/NFC, ZIGBEE®, Z-WAVE®) may be used for short-range or long-range communication between the sensors and the DREAMS system 110. In some embodiments, one or a combination of the acoustic reflection measurement device 112 the positioning/movement sensor(s) 104, the pressure sensor(s) 106, and other data sources 108 may be integrated with the DREAMS system 110, and may be configured to send data to the DREAMS system 110 in real-time or near real-time. In other embodiments, one or a combination of the acoustic reflection measurement device 112 the positioning/movement sensor(s) 104, the pressure sensor(s) 106, and other data sources 108 may not be integrated with the DREAMS system 110, but may comprise one or more modular devices that gather acoustic measurement data, movement data, pressure data, and/or other physiological data, and store the data in its own local memory. The data may then be communicated to the DREAMS system 110 after connection to the DREAMS system at a later time.

According to an aspect, the DREAMS system 110 is operative or configured to analyze the physiological data obtained from the acoustic reflection measurement device 112, the positioning/movement sensor(s) 104, the pressure sensor(s) 106, and optionally the one or more other data sources 108 for identifying physiological determinants and/or events indicative of one or more sleep-related health disorders of the individual. For example, the DREAMS system 110 may perform one or more calculations, comparisons, and/or determinations using the received physiological data to detect relevant readings and/or relevant events (e.g., jaw movements, clenching of teeth, airway collapses). Identification or detection of a relevant reading or event may be determined based on whether a reading or set of readings satisfies predetermined relevance criteria. In some embodiments, the determination may be based on whether a reading or set of readings satisfies predetermined relevance criteria in association with a sleep-related health disorder. Time data associated with an identified relevant reading or event may be used to correlate the reading/event with other physiological data. For example, data associated with an airway collapse event identified in an acoustic reflection measurement reading may be correlated with a jaw position reading, pressure reading, blood-oxygen saturation level reading, respiratory effort reading, snoring/noise reading, and/or heart rate reading at the time of the airway collapse event. The correlated data may be stored in association with the relevant reading/event.

According to an embodiment, the DREAMS system 110 is further configured to receive static 3D imaging data generated by a static 3D imaging device 116 for use in generating one or more dynamic 3D visualizations for display to a user (e.g., the individual, healthcare providers). Non-limiting examples of static 3D imaging devices 116 include a cone beam computed tomography (CBCT) device, a magnetic resonance imaging (MRI) device, and an ultrasound. The static 3D imaging device 116 may be utilized in an offline process to obtain static 3D image data (e.g., visualizations (e.g., sagittal, axial, coronal, and 3D views), measurements (e.g., volume and area measurements), visual graphs) of the individual's anatomy of interest (e.g., upper airway and mandible). For example, the static 3D imaging device 116 embodied as a CBCT device may be configured to rotate over the anatomic region of interest of the individual and acquire a set of 2D images that can be digitally combined to form a 3D image.

According to an aspect, the anatomic region of interest may include one or more anatomic landmarks (e.g., the tongue, oral cavity, nasal cavity, oropharyngeal junction (OPJ), oropharynx, epiglottis, hypopharynx, velopharynx, glottis, incisive papilla, hyoid bone, mental foramen, maxillary tuberosity, mandible, teeth), which may be identified via a manual process (e.g., one or more slices or views of the static 3D image data may be displayed, and a user may visually identify the one or more particular anatomic landmarks and define (e.g., digitally place markers on) the one or more identified landmarks), an automatic process (e.g., one or more particular anatomic landmarks may be automatically detected in one or more slices or views of the static 3D image data), or a hybrid process (e.g., one or more particular anatomic landmarks may be automatically detected, and the user may manually adjust one or more of the landmarks with a GUI tool).

The static 3D image data may be formatted based on a standard format protocol, such as DICOM® (Digital Imaging and Communications in Medicine) or other standard format that enables digital medical imaging information and other related digital data to be transmitted, stored, retrieved, printed, processed, and displayed. The static 3D imaging device 116 may or may not be located at the location or site of the DREAMS system 110. For example, the static 3D image data may be generated by the static 3D imaging device 116 at a location remote from the DREAMS system 110, and may be received by the DREAMS system over a network or other communications medium. According to an aspect, the DREAMS system 110 may be configured to store the static 3D imaging data in data storage. In some examples, the static 3D image data may be stored on a removable data storage device and read by the DREAMS system 110.

In some examples, the static 3D imaging device 116 may be configured to obtain the static 3D image data while the individual is in a supine position. The DREAMS system 110 may be configured to register the static 3D image data to baseline acoustical measurement data provided by the acoustic reflection measurement device 112, baseline movement data provided by the positioning/movement sensor(s) 104, and/or baseline pressure data provided by the pressure sensor(s) 106 based on one or more anatomic landmarks, and to generate a morph-able 3D model or representation of the individual's airway and/or mandible. In some examples, the DREAMS system 110 may be further configured to receive other image data (e.g., photographic images or other digital scan images of the individual) from one or more other image data sources 118, wherein the other image data may be superimposed on the 3D representation(s) of the individual's airway and/or mandible and used to generate a layered 3D visualization of the individual. Based on acoustical measurement data received from the acoustic reflection measurement device 112, the movement data received from the positioning/movement sensor(s) 104, and/or the pressure data received from the pressure sensor(s) 106, the 3D visual representation may be dynamically transformed as a function of time. That is, a dynamic 3D visualization is generated that may be configured to dynamically morph or transform to correspond to movements and/or changes (e.g., shape, size, obstructions, forces) to the region of interest as determined based on the received acoustic measurement data, movement data, and/or pressure data. In some embodiments, a dynamic 3D visualization is generated in association with an identified relevant reading/event (e.g., airway collapse event, jaw movement event, jaw clenching event, respiratory effort related arousal) that may be indicative of a sleep-related disorder.

The DREAMS system 110 may comprise or be communicatively connected to one or more output devices 128 for outputting information regarding data received and/or determined by the DREAMS system 110. Examples of information output by the DREAMS system 110 may include, but are not limited to, raw data, dynamic 3D visualizations, measurements, summarized data, and identified relevant readings/events. In some embodiments, the output device 128 may be or include a display device 120 or display screen capable of displaying information to a user (e.g., the individual and/or a healthcare provider). In other embodiments, the output device 128 may be or include a speaker 122 for providing sound output. In other embodiments, the output device 128 may be or include removable memory 124 capable of storing data received and/or determined by the DREAMS system 110. In other embodiments, other output devices 126 may include a printer capable of providing information via printouts, an interface for enabling the DREAMS system 110 to transmit data received and/or determined by the DREAMS system 110 to another computer system or device via a wire line connection, a wireless connection, or a subsequent coupling with the other computer system or device. According to examples, a graphical user interface (GUI) may be provided for display on the display device 120 that may be used to display information regarding data received and/or determined by the DREAMS system 110, and for enabling a user (e.g., individual/patient, healthcare provider) to interact with functionalities of the DREAMS system 110 through a manipulation of graphical icons, visual indicators, and the like. In some examples, an audible user interface (AUI) may be provided for enabling the user to interact with system 110 via voice and speech. As should be appreciated, other types of user interfaces for interacting with the DREAMS system 110 are possible and are within the scope of the present disclosure.

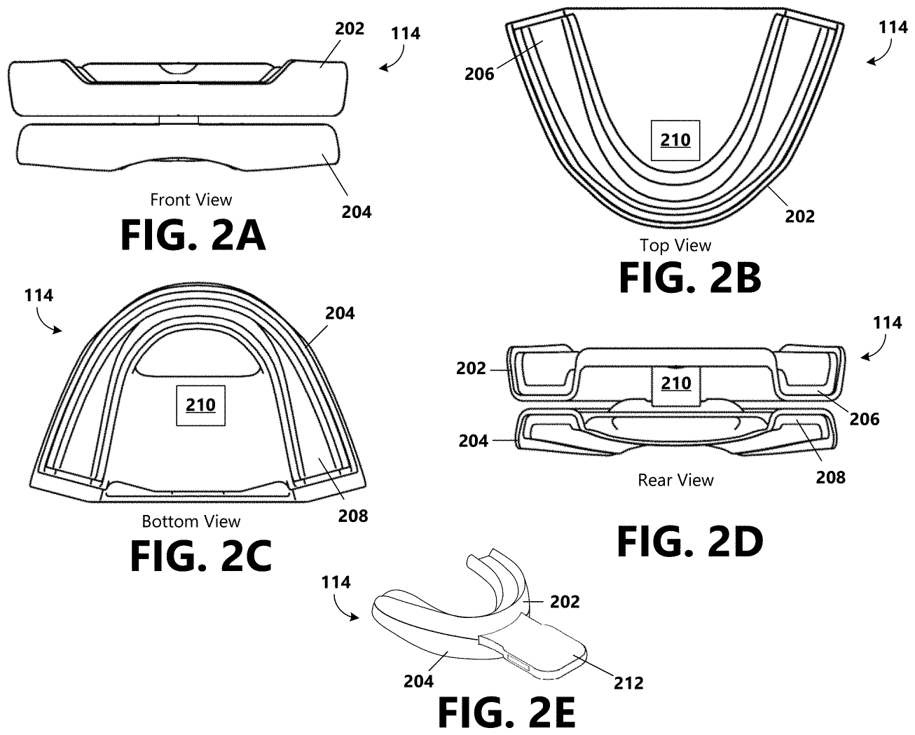

With reference now to FIGS. 2A-2E, various views of an example oral appliance device 114 are shown. Embodiments of the oral appliance device 114 may include an upper arch 202 for the upper jaw and a lower arch 204 for the lower jaw. In some embodiments, the upper arch 202 and the lower arch 204 may be a single assembly. In other embodiments, the upper arch 202 and the lower arch 204 may be separate pieces. In some embodiments, the upper arch 202 and the lower arch 204 may be separate pieces that are connected together. According to an aspect, the oral appliance device 114 may allow for the upper arch 202 and lower arch 204 to slide/move during the individual's sleep. The oral appliance device 114 may further allow the individual's tongue to move during sleep.

FIG. 2A provides a front view of the oral appliance device 114, FIG. 2B provides a top view of the upper arch 202, FIG. 2C provides a bottom view of the lower arch 204, FIG. 2D provides a rear view of the oral appliance device 114, and FIG. 2E provides an isometric view of another embodiment of the oral appliance device 114. As shown in FIGS. 2B and 2D, the upper arch 202 may comprise a generally U-shaped upper-occlusal surface 206, wherein the upper-occlusal surface 206 may be configured to contact the occlusal/incisal surfaces of the individual's upper teeth. As shown in FIGS. 2C and 2D, the lower arch 204 may comprise a generally U-shaped lower-occlusal surface 208, wherein the lower-occlusal surface 208 may be configured to contact the occlusal/incisal surfaces of the individual's lower teeth. In some embodiments, the oral appliance device 114 may be configured as a disposable appliance. In some embodiments, the oral appliance device 114 may be customized to fit the individual based on a captured impression of the individual's bite.

In some embodiments, the oral appliance device 114 may be configured to include and/or operatively connect to one or more of the acoustic reflection measurement device 112, the positioning/movement sensor 104, and/or the pressure sensor 106. For example, in some embodiments, one or more components of the acoustic reflection measurement device 112, the positioning/movement sensor 104, and/or the pressure sensor 106 may be integrated in the oral appliance device 114. In some examples and as shown in FIG. 2E, the oral appliance device 114 may include an extra-oral portion 212 that may house one or more components of the acoustic reflection measurement device 112, the positioning/movement sensor 104, and/or the pressure sensor 106. In some embodiments, the oral appliance device 114 may be configured to hold or include one or more integrated circuit chips 210. For example, the one or more integrated circuit chips 210 may be configured to perform one or more of the functionalities of the acoustic reflection measurement device 112, the positioning/movement sensor 104, and/or the pressure sensor 106. As should be appreciated, the example oral appliance device 114 illustrated in FIGS. 2A-E are for purposes of illustration only. Other designs and configurations of the oral appliance device 114 are possible and are within the scope of the present disclosure.

With reference now to FIGS. 3A-F, various views and embodiments of an example acoustic reflection measurement device 112 are shown. FIG. 3A shows various components that may be included in an example acoustic reflection measurement device 112. According to an aspect, the acoustic reflection measurement device 112 comprises an acoustic wave source 302 configured to generate acoustic waveforms that are projected down the individual's airway via an acoustic wave tube 308, reflected back out, and captured/recorded by one or more acoustic sensors 304 (e.g., microphone(s)). The amplitude of the acoustic reflections of the airway may be measured based on the time of arrival at an acoustic sensor 304 and used to determine the cross-sectional areas (CSA), length, and volume of the upper airway. The acoustic reflection measurement device 112 is configured to cause the acoustic wave source 302 to continually generate sound pulses that travel along the acoustic wave tube 308 portion of the device and into the airway of the individual. In some embodiments, the acoustic wave source 302 is embodied as a piezoelectric vibration generator operative or configured to convert electrical energy into mechanical vibrations or acoustic waves at particular frequencies that are emitted through the acoustic wave tube 308 and into the individual's airway. According to an embodiment, the piezoelectric vibration generator may comprise a chip-type piezoelectric-resonator. The piezoelectric vibration generator may further include or be operatively connected to an amplifier configured to amplify the piezoelectric vibrations. For example, at least the piezoelectric vibration generator may be included in an integrated circuit chip 210 included in the oral appliance device 114. In some embodiments, the oral appliance device 114 may be designed to define at least a portion of the acoustic wave tube 308 or to define an orifice through which at least a portion of the acoustic wave tube 308 may be positioned. The diameter of the orifice through the oral appliance device 114 may correspond to the diameter of the acoustic wave tube 308.

When an acoustic wave (i.e., incident acoustic wave) generated by the acoustic wave source 302 travels along the individual's airway, a response is generated comprising a series of reflections created due to changes in acoustic impedance within the airway. The incident and the reflected acoustic waves travel through the acoustic wave tube 308 and may be recorded by the acoustic sensor(s) 304 of the acoustic reflection measurement device 112. The acoustic signals may be processed to reveal dimensions, structure, and physiological behavior of the upper airway while the individual breathes. For example, the acoustic reflection measurement device 112 may be further configured to use these signals to determine a cross-sectional area of the individual's airway along at least a portion of the length of the individual's airway. The acoustic reflection measurement device 112 may be further configured to determine a length and volume of at least a portion of the individual's airway. For example, the acoustic reflection measurement device 112 may generate an area distance curve representing at least a portion of the individual's airway, from which the minimal cross-sectional area and volume can be derived. The cross-sectional area of the individual's upper airway may be analyzed as part of determining whether the individual may have certain sleep-related health conditions/disorders, such as apneas.

In some examples, the acoustic reflection measurement device 112 further includes a barometric sensor 320 configured to obtain air pressure readings. For example, an air pressure reading may be obtained during a relevant event, such as during an apnea or hypopnea event, which when compared against ambient air pressure, can provide an indication of severity of the apnea or hypopnea event.

As mentioned previously, the acoustic measurement data may represent one or more particular anatomic landmarks included in the individual's airway, and the one or more anatomic landmarks may be automatically identified from the acoustic reflection measurements (e.g., CSA measurements, distance measurements from one or more reference points). The anatomic landmark identifications and measurements may be used as part of mapping an anatomic landmark between a baseline reading of the acoustic measurement data and the static 3D image data, and for identifying and tracking the anatomic landmark in additional acoustic measurement data received by the DREAMS system 110 while the individual is sleeping. The acoustic reflection measurement device 112 may be configured to transmit acoustic measurement data received and/or determined by the acoustic reflection measurement device 112 to the DREAMS system 110 and/or to another computer system or device via a wire line connection, a wireless connection, or a subsequent coupling with the other computer system or device.

In some embodiments and as illustrated in FIG. 3B, the acoustic reflection measurement device 112 may be connected to a computing device 310, which may include a processor, a memory, and a display, and wherein the processor may be configured to control functionalities of the acoustic reflection measurement device 112 and to make determinations based on the received acoustic measurement data. In some examples the acoustic reflection measurement device 112 may include a Pharyngometer® (ECCOVISION of North Miami Beach, Flor.) that may be communicatively connected to the DREAMS system 110. For example, the Pharyngometer® may include, in a housing 316, an acoustic wave source 302, an acoustic wave tube 308, and acoustic reflection sensor(s) 304. The housing 316 may include or connect to a reducer 312 connected to tubing 314 that may allow for the oral appliance device 114 to be separated a distance from the housing 316. For example, as it is anticipated that the individual may be in a supine position and preferably sleeping, the housing 316 may be positioned on a nearby surface or in a stand/holder, and the tubing 314 may extend and connect to the oral appliance device 114 being worn by the individual while sleeping.

In some embodiments, for improved usability during sleep, the acoustic reflection measurement device 112 may include a noise control component 306. In some examples, the noise control component 306 includes soundproofing configured to reduce external noise generated by the acoustic wave source 302. For example, the noise control component 306 includes noise-cancelling functionality to reduce unwanted acoustic wave sounds using active noise control. In other examples, the noise control component 306 may use soundproofing materials to help prevent the acoustic waves from being emitted into the ambient environment of the individual. The noise control component 306 may be included in the acoustic reflection measurement device 112 (e.g., proximate to the acoustic wave source 302). In some examples and as illustrated in FIG. 3B, the noise control component 306 may be included in an external component, such as in headphones worn by the individual.

With reference now to FIG. 3C, a top view of an example oral appliance device 114 and another example embodiment of the acoustic reflection measurement device 112 is illustrated. The oral appliance device 114 is shown in dotted lines. As mentioned previously and as illustrated in FIG. 3C, in some examples, the oral appliance device 114 may include an extra-oral portion 212 that may house one or more components of the acoustic reflection measurement device 112. For example, the acoustic wave source 302, at least a portion of the acoustic wave tube 308, a barometric sensor 320, and one or more acoustic reflection sensors 304 may be included in the extra-oral portion 212. The extra-oral portion 212 may further include a noise control component 306.

With reference now to FIG. 3D, a top view of an example oral appliance device 114 and another example embodiment of the acoustic reflection measurement device 112 is illustrated. As mentioned previously and as illustrated in FIG. 3D, in some embodiments, the oral appliance device 114 may be configured to hold or include one or more integrated circuit chips 210. The acoustic wave source 302 may be or include a chip-type piezoelectric-resonator. For example, chip-type piezoelectric-resonator may be included in the integrated circuit chip 210 included in the oral appliance device 114. The noise control component 306 may additionally be included in the integrated circuit chip 210. In some examples, the barometric sensor 320 may be included in the integrated circuit chip 210. In some examples and as shown in FIGS. 3D and 3E, the acoustic wave tube 308 and acoustic reflection sensor(s) 304 may be sized to fit in the oral appliance device 114.

In some embodiments and as illustrated in FIG. 3F, the acoustic reflection measurement device 112 may include or connect to a source box 318 that may be configured to house the acoustic wave source 302 and the noise control component 306. The source box 318 may be configured to include one or more components (e.g., the positioning/movement sensor 104 and/or the pressure sensor 106). In some examples, the source box 318 may further connect to one or more other data sources 108 (e.g., a home sleep test unit). As should be appreciated, the example acoustic reflection measurement devices 112 illustrated in FIGS. 3A-F are for purposes of illustration only. Other designs and configurations of the acoustic reflection measurement device 112 are possible and are within the scope of the present disclosure.

With reference now to FIGS. 4A-B, example embodiments of the positioning/movement sensor 104 are illustrated. As illustrated in FIG. 4A, the positioning/movement sensor 104 may include a one or more light sources 402 (e.g., a light-emitting diode (LED) or laser diode) configured to emit beams of infrared or laser light onto a target surface 410. For example, the light source(s) 402 may emit structured (e.g., patterned) light onto the target surface 410 (e.g., occlusal/incisal surfaces of the individual's teeth, a target surface included in or connected to the oral appliance device 114), and one or more optical sensor(s) 404 may be operative or configured to receive the reflected light and convert the patterns of reflected light into digital signals that can be interpreted by a digital signal processor 406. The digital signal processor 406 may be configured to detect patterns in the digital signals, determine how those patterns have moved since a previous reading, and based on the change in patterns over a sequence of readings, determine a speed and direction of movement of the teeth relative to the positioning/movement sensor 104. For example, the movement of the teeth relative to the optical sensors 404 may be determined to correspond with mandibular movements. The optical sensor(s) 404 may include a light-detecting camera, an infrared detection device, a laser detection device, a photo-detector, and/or other optical sensor capable of detecting movement of the individual's mandible in a sleeping environment. According to an example, a plurality of light sources 402 and optical sensors 404 may be used so as to optimally obtain mandibular movement information from the user. The positioning/movement sensor 104 may further include memory 408 for storing movement/movement data. The positioning/movement sensor 104 may be configured to transmit positioning/movement data (and associated timing data) received and/or determined by the positioning/movement sensor 104 to the DREAMS system 110 and/or to another computer system or device via a wire line connection, a wireless connection, or a subsequent coupling with the other computer system or device.

In some examples and as illustrated in FIG. 4A, the one or more light sources 402 may be located in or positioned above the lower arch 204 of the oral appliance device 114, and the target surface 410 may be one or a plurality of the occlusal/incisal surfaces of the individual's lower teeth. For example, the one or more light sources 402 may be configured to emit beam of infrared or laser light onto the occlusal/incisal surfaces of the individual's lower teeth. In the example illustrated in FIG. 4A, the digital signal processor 406 and memory 408 are shown included in an intra-oral portion of the oral appliance device 114. For example, the digital signal processor 406 and memory 408 may be included in an integrated electronic chip, such as the one or more integrated circuit chips 210 included in the oral appliance device 114. In other examples and as illustrated in FIG. 4B, one or more components of the positioning/movement sensor 104 may be included in an extra-oral portion 212 of the oral appliance device 114. For example, the extra-oral portion 212 of the oral appliance device 114 may be comprised of two assemblies: a first assembly that may include at least the light source(s) 402 and the optical sensor(s) 404, and a second assembly that includes the target surface 410. For example, the first assembly may be attached to the upper arch 202 of the oral appliance device 114, and the second assembly may be attached to the lower arch 204 of the oral appliance device 114, wherein motion or movement of the individual's mandible may cause the lower arch 204 to move relative to the motion/movement, and wherein the position and movement of the mandible can be determined based on an analysis of the reflections of infrared or laser light from the target surface 410. As should be appreciated, other types of positioning/movement sensors 104 operative to determine mandibular movements and to provide corresponding movement data and timing data to the DREAMS system 110 are possible and are within the scope of the present disclosure.

With reference now to FIGS. 5A-5G, example embodiments 500a-c of the pressure sensor 106 are illustrated. FIG. 5A is a block diagram showing components of an example pressure sensor 106, FIGS. 5B and 5C provide a top view and rear view, respectively, of an example embodiment 500a of the pressure sensor 106 included in an example oral appliance device 114, FIGS. 5D and 5E provide a bottom view and a rear view, respectively, of another example embodiment 500b of the pressure sensor 106 included in the lower arch 204 of an example oral appliance device 114, and FIGS. 5F and 5G provide a top view and rear view, respectively, of another example embodiment 500c of the pressure sensor 106 included in an example oral appliance device 114. According to an aspect and with reference to FIG. 5A, the pressure sensor 106 may include one or more pressure sensing elements 502 operative or configured to continually obtain pressure data readings (e.g., static and dynamic occlusion/masticatory forces) that may be recorded and transmitted to the DREAMS system 110. In some examples, the pressure sensing element(s) 502 may be configured as piezoelectric sensors that use the piezoelectric effect to measure changes in static or dynamic occlusion/masticatory forces. For example, the pressure sensing element(s) 502 may include electrically conductive material arranged on a substrate, wherein the conductive material may comprise piezoresistive (pressure sensitive) ink material. Compression force or other mechanical stress of the materials may alter the electrical properties of the ink and thus increase or decrease the electrical resistance in the piezoresistive ink material. Due to the change in resistance, an output voltage may be produced that is proportional to the sensed pressure. In other examples, the pressure sensing element(s) 502 may include a matrix of capacitive sensing elements comprising arrays of conductive strips separated by a thin compressible elastomer dielectric. Pressure applied to the surface of the pressure sensing element 502 may compress the dielectric, which may result in a change in the voltage across the capacitive element.

The produced voltage signals may be provided to a processor 506 and/or stored in memory 508. The voltage signals may be converted into pressure readings that may be evaluated and stored as parameter values for the occlusal/masticatory forces. As should be appreciated, other types of pressure sensing elements 502 configured to obtain pressure readings associated with occlusal/masticatory forces may be used and are within the scope of the present disclosure. The processor 506 and memory 508 may be operatively connected to the pressure sensing element(s) 502. In some examples and as shown in FIG. 5C, the processor 506 and memory 508 may be included in an integrated electronic chip, such as the one or more integrated circuit chips 210 included in the oral appliance device 114. In other examples and as illustrated in FIG. 5D, the processor 506 and memory 508 of the pressure sensor 106 may be included in an extra-oral portion 212 of the oral appliance device 114. The pressure sensor 106 may be configured to transmit pressure data (and associated timing data) received and/or determined by the pressure sensor 106 to the DREAMS system 110 and/or to another computer system or device via a wire line connection, a wireless connection, or a subsequent coupling with the other computer system or device.

In one embodiment 500a and as illustrated in FIGS. 5B and 5C, at least one pressure sensing element 502 may be disposed in the upper arch 202 of the oral appliance device 114, wherein pressure applied to the upper-occlusal surface 206 of the upper arch 202 may be sensed by the pressure sensor 106. For example, the oral appliance device 114 may be designed to hold the at least one pressure sensing element 502 on the upper-occlusal surface 206 of the upper arch 202. In another example, the at least one pressure sensing element 502 may be positioned in the upper arch 202 and inserted in the upper-occlusal surface 206 or between the upper-occlusal surface 206 and the upper arch 202.

In another embodiment 500b and as illustrated in FIGS. 5D and 5E, at least one pressure sensing element 502 may be disposed in the lower arch 204 of the oral appliance device 114, wherein pressure applied to the lower-occlusal surface 208 of the lower arch 204 may be sensed by the pressure sensor 106. For example, the oral appliance device 114 may be designed to hold the at least one pressure sensing element 502 on the lower-occlusal surface 208 of the lower arch 204. In another example, the at least one pressure sensing element 502 may be positioned in the lower arch 204 and inserted in the lower-occlusal surface 208 or between the lower-occlusal surface 208 and the lower arch 204.

In another embodiment 500c and as illustrated in FIGS. 5F and 5G, at least a portion of the pressure sensing element 502 may be sandwiched between the upper arch 202 and the lower arch 204 of the oral appliance device 114. For example, the oral appliance device 114 may be designed to hold the pressure sensing element 502 between the upper arch 202 and the lower arch 204, wherein pressure applied to the upper-occlusal surface 206 of the upper arch 202 or to the lower-occlusal surface 208 of the lower arch 204 may be sensed by the pressure sensor 106. One or a combination of these embodiments 500a-c of the pressure sensor 106 may be used in or in operative connection with the oral appliance device 114. As should be appreciated, other types and configurations of the pressure sensor 106 operative to obtain pressure data related to occlusal/masticatory forces and to provide corresponding pressure data and timing data to the DREAMS system 110 are possible and are within the scope of the present disclosure.

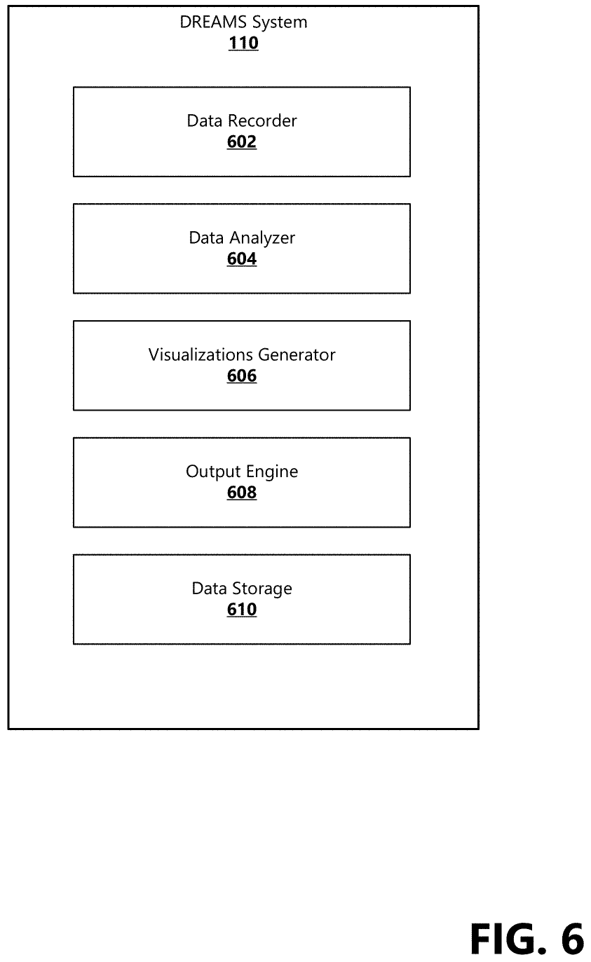

FIG. 6 is a block diagram showing various components of an example DREAMS system 110. With reference now to FIG. 6, the example DREAMS system 110 may include a data recorder 602, a data analyzer 604, a visualizations generator 606, an output engine 608, and data storage 610. As should be understood by those skilled in the art, one or more of the components (e.g., data recorder 602, a data analyzer 604, a visualizations generator 606, an output engine 608, and data storage 610) can be integrated or provided in any combination of separate systems, wherein FIG. 6 shows only one example. Generally, the various components of the DREAMS system 110 are configured to collect physiological parameter data of an individual while sleeping, analyze the collected data, and generate and provide results, including information regarding data received and/or determined by the system, for use in a variety of clinical and/or user-education applications. Example results include graphs, dynamic 3D models, measurements, summarized information about the collected data and about events that may be indicative of a sleep-related disorder, etc. Example clinical applications can include diagnosing an individual with a sleep-related disorder, determining efficacy of a treatment and/or therapy, verifying effectiveness of a rendered treatment and/or therapy, and other clinical applications.

The data recorder 602 is illustrative of a software application (being executed on a computer or microprocessor), module, or computing device operative or configured to receive physiological data obtained and/or determined by various data sources. The physiological data may include timing and measurement data of at least a portion of the individual's airway, mandibular movement data, bite force data, as well as other physiological data associated with determinants of various sleep-related disorders (e.g., breathing disorders, movement disorders, other disorders). For example, the various data sources may include one or a combination of: the acoustic reflection measurement device 112, the positioning/movement sensor 104, the pressure sensor 106, and other data source(s) 108, such as a home sleep test unit, a polysomnogram unit, and/or one or more other sensors configured to obtain physiological data of the individual during sleep. In some examples, the data recorder 602 is configured to receive physiological data obtained and/or determined by one or more of the data sources continually. For example, as data are obtained and/or determined by a data source, the data source may transmit the data to the DREAMS system 110 via a wired and/or wireless connection, and the data recorder 602 may receive the data in real-time or near real-time. In other examples, the data recorder 602 may be configured to receive physiological data obtained and/or determined by one or more of the data sources in batches. For example, a data source may locally store data obtained and/or determined by the data source, and may transmit the data in a batch to the DREAMS system 110 after connection (e.g., wired and/or wireless) to the DREAMS system at a later time. According to an aspect, the data recorder 602 may be further configured to store the received data in the data storage 610.

The data analyzer 604 is illustrative of a software application (being executed on a computer or microprocessor), module, or computing device operative or configured to analyze the received data to determine relevant readings and events that may be indicative of a sleep-related disorder (e.g., apneas, hypo-apneas, mixed apneas, RERAs, sleep bruxism, obstructed airway-associated head/neck movement disorders, TMD). For example, the data analyzer 604 may be operative to perform measurements, calculations, comparisons, and/or make determinations using the received physiological data to detect relevant readings and/or relevant events, which may include, but are not limited to, jaw movements, clenching of teeth, airway collapses, elevated heart rate, low blood oxygen saturation levels, etc. In some examples, the data analyzer 604 may be configured to perform one or more processing operations to the received data. For example, processing the data may include one or a combination of: converting the data, packaging the data, validating the data, combining the data, enhancing the data, and sorting the data, among other data processing operations. According to one example, the data analyzer 604 may be configured to receive acoustic measurement readings in a raw format, and processing the readings may provide an area distance curve representing the individual's airway from which minimal cross-sectional area and volume can be derived and used in an analysis of the airway and in comparison with other collected data.

Identification or detection of a relevant reading or event may be determined based on whether a reading or set of readings satisfies predetermined relevance criteria. In some embodiments, the predetermined relevance criteria may be associated with an event associated with a sleep-related health disorder. For example, changes in acoustic measurement readings of a portion of the individual's airway may be determined to satisfy relevance criteria/rules associated with an airway collapse event (e.g., relevant event). As an example, a relevance rule associated with an apnea event may be defined as a cessation of air flow for at least N seconds (e.g., 10 seconds). As another example, a relevance rule associated with a hypopnea event may be defined as reduced air flow (e.g., of at least 30% from baseline) for at least N seconds (e.g., 10 seconds). As another example, a relevance rule associated with an obstructive respiratory event may be defined as a detection (from an evaluation of the collected data) of certain activities, such as snoring, thoracoabdomnial paradox, increased respiratory effort, etc. In some examples, the data analyzer 604 may be configured to determine a severity score for a relevant event according to a set of rules. For example, a severity score may be based on a severity assessment (e.g., one or a combination of: measurement values, a rate of occurrence, and duration) of a reading or a set of readings. In one example, a severity assessment for an apnea or hypopnea event may include an analysis of an air pressure reading obtained by the barometric sensor 320 during the apneas or hypopnea. A severity score may include various levels of severity (e.g., normal, mild, moderate, or severe). In some examples, the data analyzer 604 may be configured to determine a confidence score/level for a determination (e.g., determination of a relevant event, determination of a severity score). In some examples, a diagnosis of a sleep-related health disorder may be based in part on determined confidence scores/levels.

According to an aspect, the data analyzer 604 may be further configured to correlate readings from two or more data sources for determining relationships between two or more variables (e.g., readings from two or more data sources). Time data associated with readings or determined events may be used to correlate a reading/event with other physiological data. For example, based on time, a cross-sectional area measurement associated with an airway collapse event identified in an acoustic reflection measurement reading may be correlated with a jaw position reading, pressure reading, air pressure reading, blood-oxygen saturation level reading, respiratory effort reading, snoring/noise reading, and/or heart rate reading at the time of the airway collapse event. A strength and direction (e.g., positive or negative) of the relationship between at least two variables (e.g., the cross-sectional area measurement and the jaw position) may be determined based on correlation values. In some examples, a scatter plot and regression analysis may be used to determine correlation values. The correlated data may be stored in the data storage 610 in association with a determined relevant reading/event.

In some examples, the data analyzer 604 may be further configured to validate data to ensure accuracy according to a set of rules. For example, an analysis of the correlated data may reveal one or more readings that fall outside the overall pattern of an identified relationship between variables. These readings may be determined as anomalies or exceptions in the data, and may be excluded in order to obtain a better assessment of the correlation between the variables. For example, an anomaly may be associated with sleep talking, coughing, sensor disruption, or other activities that may not be relevant to evaluation of the individual in association with a sleep-related disorder.

The visualizations generator 606 is illustrative of a software application (being executed on a computer or microprocessor), module, or computing device operative or configured to generate visualizations of the received and/or determined data. In some examples, the visualizations generator 606 may generate 2D and/or 3D visualizations of one or more of the acoustic measurement data, positioning/movement data, pressure data, and home sleep test/polysomnogram data. In some examples, the visualizations generator 606 may generate one or more 3D dynamic visualizations based on physiological data received from the one or more data sources and based on received image data (e.g., static 3D image data, photographs, oral scans, ultrasounds, radiographs). For example, the one or more 3D dynamic visualizations may show movements of the individual's airway, positioning and/or dynamic movements of the individual's mandible, and/or occlusal force distributions.

In some embodiments, the visualizations generator 606 may be, include, or be configured to perform functionalities similar to the 3D Rendering and Enhancement of Acoustic Data (READ) system described in co-pending provisional application U.S. 62/955,657 titled “Dynamic 3-D Anatomical Mapping and Visualization” filed Dec. 31, 2019, which is incorporated herein by reference. For example, the visualizations generator 606 may be configured to receive static 3D image data representing an anatomic region of interest (e.g., at least the individual's airway and/or mandible), receive acoustic measurement data representing at least a portion of the anatomic region of interest (e.g., airway), receive position/movement data representing at least a portion of the anatomic region of interest (e.g., mandible), map the acoustic measurement data and/or position/movement data to the static 3D image data based on one or more anatomic landmarks, and generate a dynamic 3D visualization of the anatomic region by transforming the 3D visualization based on the acoustic measurements and/or position/movement readings associated with the one or more anatomic landmarks.

As part of mapping the acoustic measurement data to the static 3D image data, the visualizations generator 606 may map/register one or more anatomic landmarks associated with the anatomic region of interest included in a first set (e.g., baseline reading) of acoustic measurement data to one or more corresponding anatomic landmarks identified in the static 3D image data. In some aspects, multi-planar visualization of the static 3D image data may be used to identify the one or more particular anatomic landmarks of at least a portion of the individual's airway in the static 3D image data. In some examples, the baseline reading may be acquired while the individual is in a supine position. For example, the supine position may be similar to a sleeping position of the individual during data collection by the acoustic reflection measurement device 112, the positioning/movement sensor(s) 104, the pressure sensor(s) 106, and the one or more other data sources 108. In some examples, the baseline reading may be acquired while the individual is performing a respiration procedure, such as a Muller's maneuver, where the airway may be collapsed responsive to, after a forced expiration, an attempt at inspiration made with a closed mouth and nose (or closed glottis). As part of mapping the position/movement data to the static 3D image data, the visualizations generator 606 may map/register one or more anatomic landmarks associated with the anatomic region of interest included in a first set (e.g., baseline reading) of position/movement data to one or more corresponding anatomic landmarks identified in the static 3D image data. In some examples, the individual may use a head positioning device and/or a mandibular positioning device during the capture of the baseline acoustic measurement data, the baseline positioning/movement data, and/or the 3D image data to allow for similar positioning for more accurate registration of anatomic landmarks between data sets. By mapping the one or more anatomic landmarks between the baseline acoustic measurement data and the static 3D image data and between the baseline positioning/movement data and the static 3D image data, the DREAMS system 110 may be enabled to identify and track changes in measurements/movements in association with the anatomic landmarks in the acoustic measurement data and positioning/movement data collected while the individual is sleeping. In some examples, one or more sets of other image data from one or more other image data sources 118 may be received by the DREAMS system 110 and registered to the static 3D image data based on the one or more anatomic landmarks. For example, the one or more sets of other image data may be superimposed on the 3D representation of the individual's airway and/or mandible and used to generate a layered 3D visualization of the individual.

The visualizations generator 606 may be further operative or configured to generate one or more morph-able 3D models representing at least the anatomic region of interest (e.g., at least a portion of the individual's airway, the mandible) based on the static 3D image data, the baseline acoustic measurement data, and/or the baseline positioning/movement data. In some examples, the morph-able 3D model may be automatically generated. In other examples, the morph-able 3D model may be generated in response to a determination of a relevant reading or event. In other examples, the morph-able 3D model may be generated in response to a user request. According to an aspect, the visualizations generator 606 may be configured to generate the morph-able 3D model using CAD functionalities. One example embodiment of the visualizations generator 606 may be configured to access a 3D view or model of the individual's airway included in the static 3D image data and convert the 3D view or model into the morphable 3D model that includes the identified/mapped anatomic landmarks. Another example embodiment of the visualizations generator 606 may be configured to generate the morph-able 3D model using CAD functionalities based on various views (e.g., sagittal, axial, coronal) of the individual's airway and mandible included in the static 3D image data. According to an aspect, the visualizations generator 606 may be configured to link the determined mappings between the one or more anatomic landmarks in the baseline data and the static 3D image data to the one or more anatomic landmarks in the morphable 3D model. According to another embodiment, the visualizations generator 606 may be configured to generate a 3D representation of the anatomic region of interest based on the baseline acoustic measurement data, and superimpose this 3D representation with a 3D view of the anatomic region of interest included in the static 3D image data based on the one or more identified/defined anatomic landmarks to generate the morph-able 3D model.

In example aspects, the visualizations generator 606 may be further operative or configured to transform the morph-able 3D model based on received acoustic measurement data for providing a dynamic 3D visualization of at least the individual's airway. For example, the received acoustic measurement data may include updated measurements/positions relative to the one or more anatomic landmarks. Based on the mappings to the landmarks, the morph-able 3D model may be transformed to represent the updated measurements/positions. For example, a first visualization of at least the airway provided by the morph-able 3D model may be transformed into a next visualization to correspond with the determined measurements of the airway based on the location and measurements related to the anatomic landmarks. Accordingly, a dynamic 3D visualization of at least the individual's airway is provided. In some embodiments, the received acoustic measurement data are measurements associated with determined relevant events, and the morph-able 3D model may be transformed to represent the relevant events. For example, a dynamic 3D visualization may be generated for one or more of the relevant events.

In some example aspects, the visualizations generator 606 may be further operative or configured to transform the morph-able 3D model based on positioning/movement data for providing a dynamic 3D visualization of at least the individual's mandible. For example, the received acoustic measurement data may include updated measurements/positions relative to one or more anatomic landmarks. Based on the mappings to the landmarks, the morph-able 3D model may be transformed to represent the updated measurements/positions. For example, a first visualization of at least the mandible provided by the morph-able 3D model may be transformed into a next visualization to correspond with the positions/movements of the mandible based on the location and measurements related to the anatomic landmark(s). Accordingly, a dynamic 3D visualization of at least the individual's mandible is provided. In some embodiments, the received positioning/movement data are measurements associated with determined relevant events, and the morph-able 3D model may be transformed to represent the relevant events. For example, a dynamic 3D visualization may be generated for one or more of the relevant events.

In example aspects, the morph-able 3D model may be transformed to represent the dynamics of a combination of the individual's airway, the individual's mandible, and/or the individual's occlusal forces. For example, the acoustic reflection measurement data associated with a relevant event, the positioning/movement data associated with the same relevant event may be correlated and visually represented by a dynamic 3D visualization. In some examples, the dynamic 3D visualizations may provide a dynamic visualization of positions/movements of additional anatomy, such as of the individual's tongue, the hyoid bone, etc. According to an embodiment, one or more transformations of the morph-able 3D model (i.e., a dynamic 3D visualization) may be recorded and stored in the data storage 610. The recording of the dynamic 3D visualization may be played back and displayed on a display device 120.

In some embodiments, the data storage 610 may further include a knowledge database comprising a plurality of datasets of static 3D images (e.g., various views of CBCT images and associated measurement data) of at least airways and mandibles of the individual and/or of various individuals. For example, the images included in the knowledge database may include the one or more anatomic landmarks, and can be used by the visualizations generator 606 as references to the geometries/positions of portions of the airway or mandible in association with various measurements/positions related to one or more anatomic landmarks. Accordingly, the static 3D image data included in the knowledge database may be used as a target image in a next visualization (i.e., morphing of the 3D model) to correspond with geometries/positions of portions of the airway or mandible based on the acoustic measurement data and/or positioning/movement data. In example aspects, the visualizations generator 606 may animate the transformation between a first visualization and the next to simulate actual movement, shape, and obstructions of the individual's anatomy. In some embodiments, colorization may be used by the visualizations generator 606 to reflect changes in measurements of the individual's anatomy in relation to one or more anatomic landmarks. In some examples, the DREAMS system 110 may further include measurement tools configured to measure distances, diameters, etc., of anatomy represented in the static 3D image data, the acoustic measurement data, and/or in the generated 3D model.

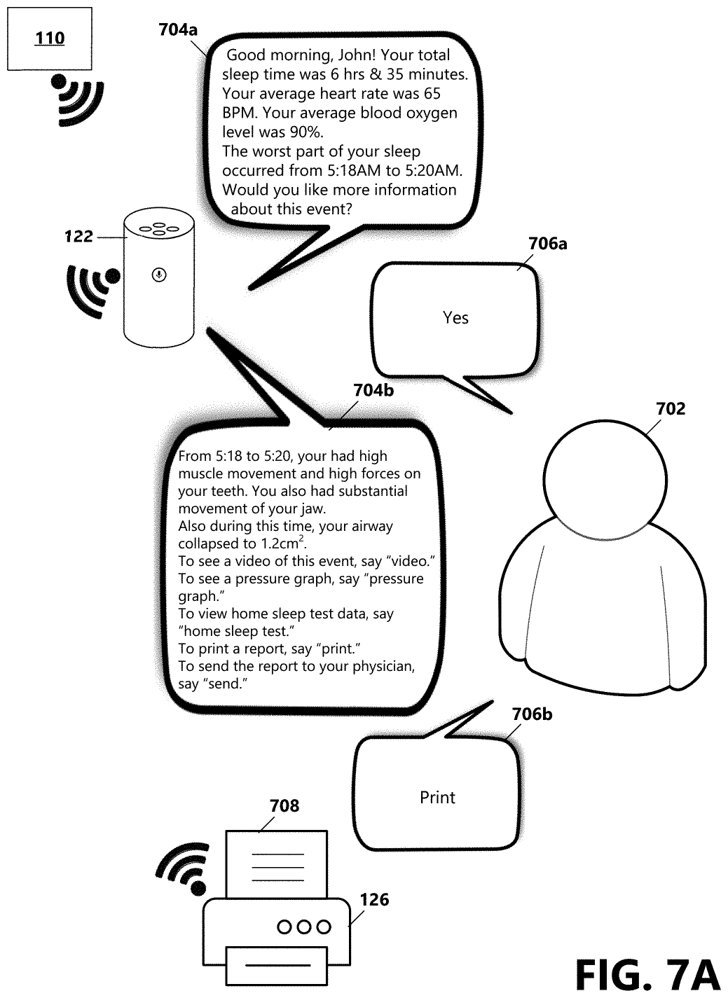

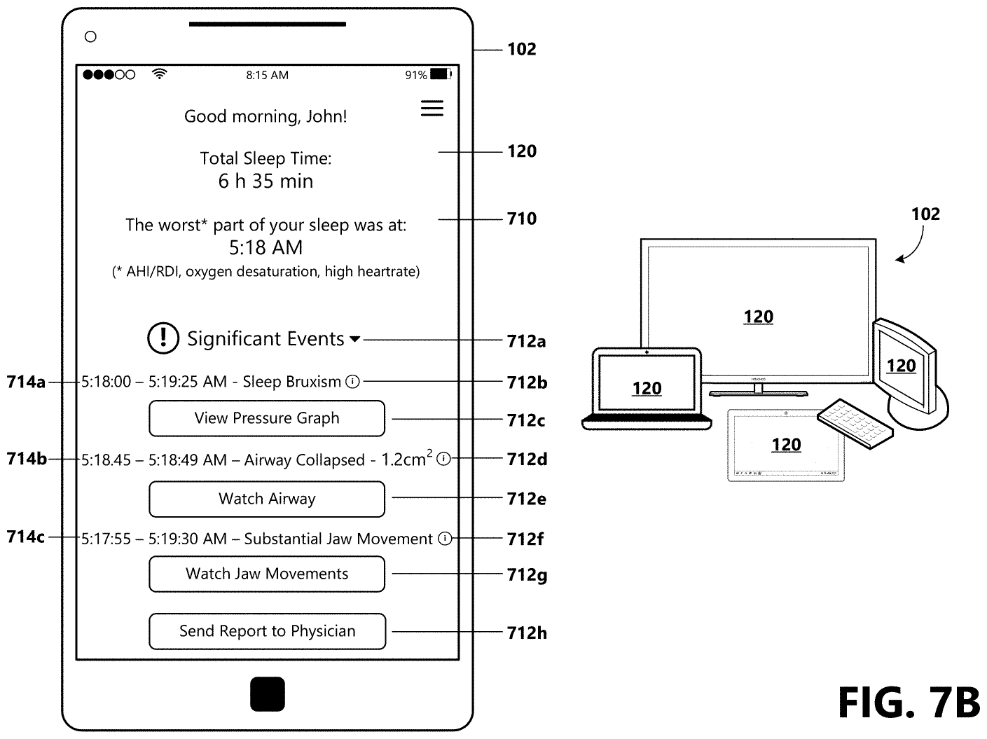

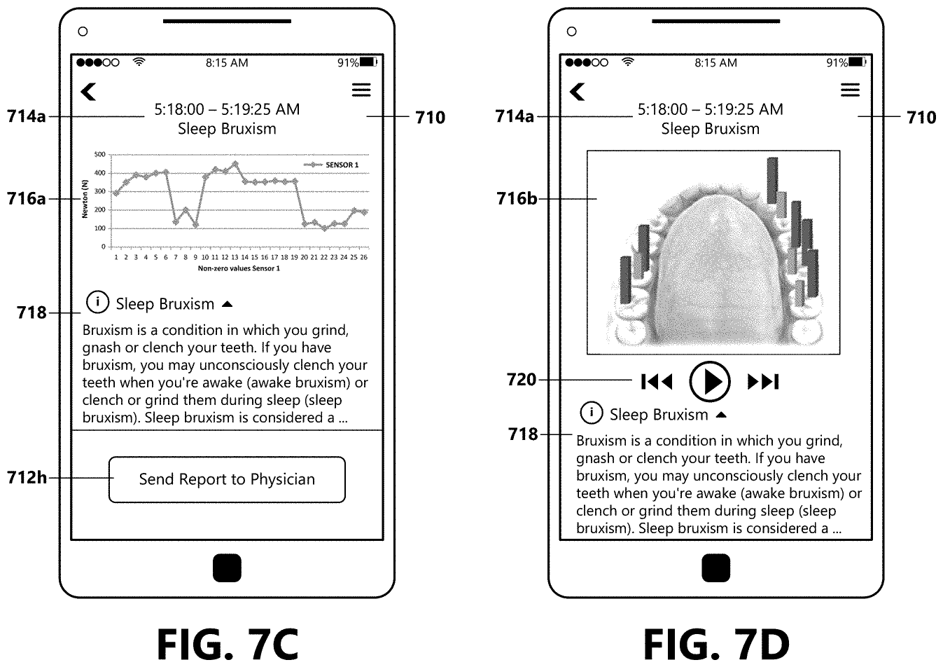

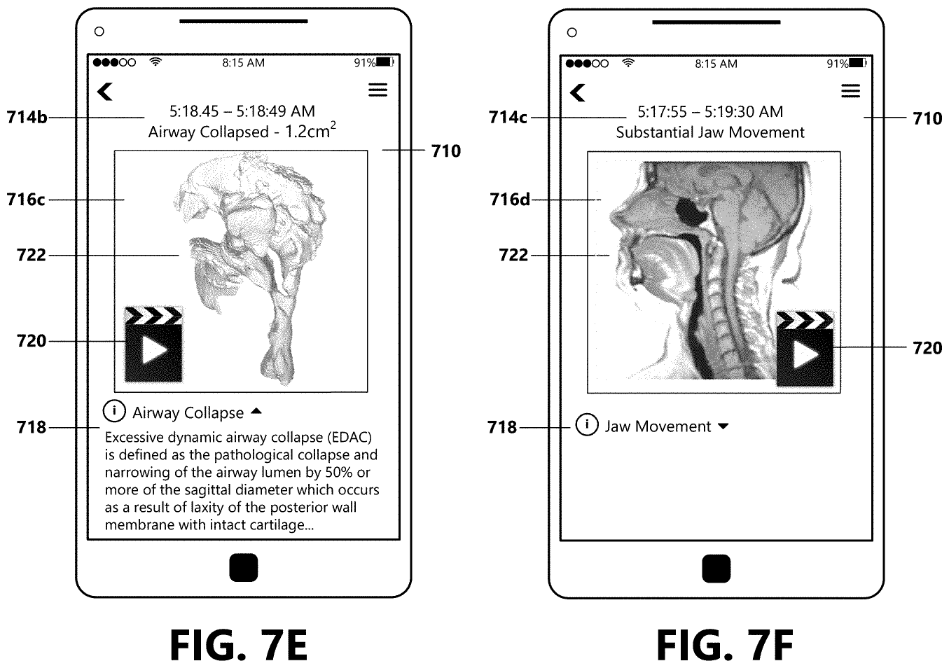

The output engine 608 is illustrative of a software application (being executed on a computer or microprocessor), module, or computing device operative or configured to outputting information regarding data received and/or determined by the DREAMS system 110 to one or more output devices 128. Examples of information output by the DREAMS system 110 may include summarized information, dynamic 3D visualizations, and data (e.g., raw data, determined data, measurements) related to identified relevant events during the individual's sleep cycle(s). In some embodiments, the output engine 608 may be configured to provide a user interface via which information received and/or determined by the DREAMS system 110 may be displayed on a display device 120 (e.g., a graphical user interface) or played audibly via a speaker 122. The user interface may enable a user (e.g., individual/patient, healthcare provider) to interact with functionalities of the DREAMS system 110 through a manipulation of graphical icons, visual indicators, and the like. In some examples, an audible user interface (AUI) may be provided for enabling the user to interact with system 110 via voice and speech recognition. Examples of user interfaces and information that may be output by the DREAMS system 110 are illustrated in FIGS. 7A-E and are described below.

In some embodiments, the output device 128 may be or include removable memory 124 capable of storing data received and/or determined by the DREAMS system 110, and the output engine 608 may be configured to save data received and/or determined by the DREAMS system 110 to the removable memory. In some embodiments, the DREAMS system 110 may be configured to interface a printer capable of providing information via printouts. In some embodiments, the DREAMS system 110 may be configured to transmit data received and/or determined by the DREAMS system 110 to another computer system or device via a wire line connection or a wireless connection. In some embodiments, the DREAMS system 110 may be further configured to convert one or more 3D visualizations generated by the visualizations generator 606 into various file formats for output to other systems or devices. For example, a visualization may be converted into a universally accepted 3D file format, such as standard tessellation language (STL) or wavefront object (OBJ), which can be output to a 3D printer.

FIGS. 7A-E illustrate various examples of user interfaces and information that may be output by the DREAMS system 110. With reference to FIG. 7A, according to an aspect, the DREAMS system 110 may be in communication with (e.g., via a wired connection or wirelessly connected to) one or more output devices 128, such as a speaker 122 and a printer (e.g., other output device 126). For example, the speaker 122 may be a standalone device (connected to a network) or may be connected to or integrated with a computing device 102. An AUI may be provided, via the speaker 122, with which the user 702 may interact with the DREAMS system 110 via voice recognition or speech commands. Responsive to a user input, such as an indication of a selection to receive output from the DREAMS system 110, the speaker 122 may provide audible output 704a,b of data received and/or determined by the DREAMS system 110. In some examples, the output (e.g., audible output 704, visual output) may include measurements, summarized data of one or more of the acoustic reflection measurement data, positioning/movement data, pressure data, or other data (e.g., home sleep test data, polysomnogram data). For example, the summarized data may include totals, averages, and/or extremas (e.g., maximums and/or minimums) of the received physiological data. In some examples, the output (e.g., audible output 704, visual output) may include summarized data about one or more determined relevant events.

In some examples, the output (e.g., audible output 704, visual output) may include options for the user 702 to interact with the DREAMS system 110. For example, the user 702 may be prompted for a response (e.g., to request details about the summarized data, measurements or additional information, or for the system to perform an action). The DREAMS system 110, via the speaker 122, may receive user responses 706a,b. For example and as illustrated, the user 702 may provide a user response 706a indicating a request for more information about the summarized data, and the DREAMS system 110, via the speaker 122, may provide an audible response (i.e., audible output 704) including the requested information. As another example and as illustrated, the user 702 may provide a user response 706b indicating a request for the DREAMS system 110 to perform an action, such as to print a report 708 including information regarding data received and/or determined by the DREAMS system 110. Another action may include sending a report 708, including information regarding data received and/or determined by the DREAMS system 110, to a healthcare professional (e.g., via an email communication, a portal, facsimile, or other HIPAA-compliant method). Other actions are possible and are within the scope of the present disclosure.

With reference now to FIG. 7B, according to an aspect, the DREAMS system 110 may be in communication with (e.g., via a wired connection or wirelessly connected to) a display device 120 or one or more computing devices 102 that include a display 120 via which the system can provide visual output to a user 702. For example, the DREAMS system 110 may generate and provide a graphical user interface (GUI) 710 configured to display information regarding data received and/or determined by the DREAMS system 110 and to provide graphical elements 712 that enable the user 702 to interact with the DREAMS system 110 via interactions with the graphical elements via various input methods (e.g., pointing device, touch, speech, gestures). In some examples, the GUI 710 includes a menu or listing of various options, for viewing data collected by and/or determined by the DREAMS system 110. For example, the various options can include options to view information about and/or visualization(s) of one or more relevant events determined by the DREAMS system 110. According to an aspect, the visualization(s) may include dynamic 3D visualizations based on one or a combination of physiological data and image data from various data sources. For example, various physiological data may be correlated based on time of a determined relevant event, and the dynamic 3D visualization may represent dynamics of the individual's anatomy in association with the relevant event.

The example GUI 710 illustrated in FIG. 7B includes a display of visual output including summarized data, and further includes a listing of determined relevant events 714. One or more graphical elements 712a-h (generally, 712) may be included that, when selected, provide additional information about the relevant events 714a-c (generally, 714). In some examples, responsive to a selection of a particular graphical element 712c, e,g associated with a relevant event 714, the DREAMS system 110 may generate and provide a visualization representing the relevant event 714. In other examples, the DREAMS system 110 may generate one or more visualizations of one or more relevant events 714 prior to receiving a selection to view a visualization of the event. In some examples, a graphical element 712h may be provided, which when selected, sends a request to the DREAMS system 110 to perform an action, such as to send a report 708, including information regarding data received and/or determined by the DREAMS system 110, to a healthcare professional (e.g., via an email communication, a portal, facsimile, or other HIPAA-compliant method).

With reference now to FIG. 7C, a first example visualization 716a representing a first relevant event 714a is illustrated. For example, the first relevant event 714a may be associated with sleep bruxism, and the first example visualization 716a may be a 2D visualization, such as a line graph representing the movements/forces as a function of time associated with the sleep bruxism event. As an example, responsive to receiving a selection of the graphical element 712c associated with the first relevant event 714a, the GUI 710 may be updated to display the first visualization 716a. In some examples, additional information 718 about the relevant event 714 may also be provided.

With reference now to FIG. 7D, a second example visualization 716b representing the first relevant event 714a is illustrated. For example, the second example visualization 716b may be a 3D visualization associated with the sleep bruxism event. In some examples, the 3D visualization may be a dynamic visualization that may be based on received pressure data and that may be played to show occlusal force changes during the signification event 714a. In some examples and as illustrated, the visualization 716b may be overlaid/superimposed onto one or more images received in image data provided by one or more imaging data sources 118 to show occlusal force changes in relation to the individual's anatomy (e.g., teeth). When a dynamic visualization 716b is provided, playback controls 720 may also be provided to control playback of the dynamic visualization.

With reference now to FIG. 7E, a third example visualization 716c representing a second relevant event 714b is illustrated. For example, the second relevant event 714b may be associated with an airway collapse event, and the third example visualization 716c may be a dynamic 3D visualization representing the airway collapse event. The dynamic 3D visualization may include the morph-able 3D model 722 described above. For example, the morph-able 3D model 722 may be generated based on a CBCT scan of the individual. Playback of the dynamic 3D visualization 716c may show the 3D model 722 morphing to represent dynamic movements of the individual's airway based on received acoustic reflection measurement data.

With reference now to FIG. 7F, a fourth example visualization 716d representing at least a third relevant event 714c is illustrated. For example, the third relevant event 714c may be associated with jaw movement, and the fourth example visualization 716d may be a dynamic 3D visualization representing the jaw movement event. The dynamic 3D visualization may include the morph-able 3D model 722 described above. For example, the morph-able 3D model 722 may be generated based on a CBCT scan of the individual. Playback of the dynamic 3D visualization 716d may show the 3D model 722 morphing to represent dynamic movements of the individual's mandible based on received positioning/movement data. In some examples, a dynamic 3D visualization 716 may represent a plurality of relevant events 714. For example, the morph-able 3D model 722 included in the fourth example visualization 716d may additionally morph during playback to represent dynamic movements of the individual's airway at the same time as the third relevant event 714c based on received acoustic reflection measurement data. In other examples, additional visualizations associated with one or more other relevant events 714 occurring at the same time as the third relevant event 714c may be overlaid on the visualization 716. For example, another 3D visualization based on received pressure data may be included that shows occlusal force changes during the third relevant event 714c. As should be appreciated, other GUI 710 designs, other graphical elements, other types of relevant events 714, and other visualizations 716 are possible and are within the scope of the present disclosure.

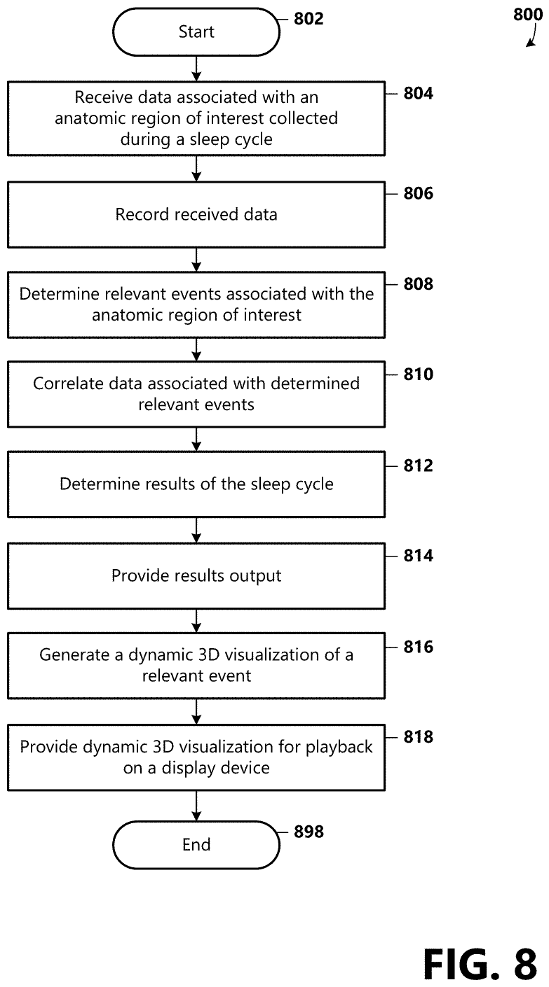

FIG. 8 is a flow diagram depicting general stages of an example process for providing sleep-related disorder data collection, assessment, and visual representation. With reference now to FIG. 8, the method 800 starts at OPERATION 802 and proceeds to OPERATION 804, where the DREAMS system 110 receives physiological data associated with an anatomic region of interest of an individual collected during one or more sleep cycles; wherein, in some examples, a sleep cycle may be a full sleep period comprised of a plurality of sleep cycles. As should be appreciated, aspects are configured to collect various physiological data, including acoustic reflection measurement data, while the individual is sleeping, which is an improvement over current technologies, which do not provide for such data collection. For example, current acoustic reflection measurement devices are not practical for overnight data collection due at least in part to the size and the loudness of current technologies. Aspects of the present disclosure enable the collection of various data via a noninvasive oral appliance device 114 (e.g., does not include a large wave tube portion, is not loud due to noise control, does not require use of a mask) configured to be worn during sleep, wherein the oral appliance device 114 comprises various sensors, which may be included in one or a combination of an intra-oral portion of the device and an extra-oral portion of the device.

As described above, the physiological data may be collected by a plurality of sensors/data sources and transmitted to the DREAMS system 110 in real-time, near real-time, or in one or more batches. The plurality of sensors/data sources may include one or a combination of an acoustic reflection measurement device 112, a positioning/movement sensor 104, a pressure sensor 106, and/or other data sources 108, such as a home sleep test unit or polysomnogram unit. According to an aspect, one or more of the sensors/data sources may be integrated with, included in, or attached to the oral appliance device 114 described above, which the individual may insert in his/her oral cavity prior to a sleep cycle and wear for the duration of the sleep cycle. According to an aspect, the physiological data received by the include readings/measurements collected by the one or more sensors/data sources and further include timing data associated with readings/measurements. For example, the collected data may be timestamped by the sensors/data sources or, if the data are received in real-time or near real-time, the data may be timestamped by the DREAMS system 110. The collected physiological data may include acoustic reflection measurement data, positioning/movement data, pressure data, and in some examples, data related to the individual's blood-oxygen saturation levels, airflow, respiratory effort, heart rate, heart rhythm, breathing pattern, eye movement, muscle activity, brain activity, snoring and other noises made while sleeping, etc. At OPERATION 806, the received data may be stored in data storage 610.

At OPERATION 808, the collected physiological data may be processed and analyzed by the DREAMS system 110 for determining one or more relevant events 714. As described above, relevant events 714 may include, but are not limited to, jaw movements, clenching of teeth, airway collapses, elevated heart rate, low blood oxygen saturation levels, etc. For example, a relevant event 714 may be an event associated with and that may be indicative of a sleep-related disorder (e.g., apneas, hypo-apneas, mixed apneas, RERAs, sleep bruxism, obstructed airway-associated head/neck movement disorders, TMD). Determination of a relevant event 714 may be based on whether measurements/readings included in the collected data satisfy relevance criteria/rules associated with a relevant event 714. In some examples, the DREAMS system 110 may be configured to determine a severity score for a relevant event 714 according to a set of rules. In some examples, the DREAMS system 110 may be further configured to determine a confidence score for the determination of a relevant event 714. In some examples, processing the collected data may include a data cleaning operation for detecting anomalies/scatter in the data. For example, anomalies/scatter may be associated with sleep talking, coughing, sensor disruption, or other activities that may not be relevant to evaluation of the individual in association with a sleep-related disorder.

At OPERATION 810, data associated with a determined relevant event 714 may be correlated. According to an aspect, time data associated with an identified relevant reading/event may be used to correlate the event with other physiological data. For example, an analysis of positioning/movement data may be determined to be associated with a jaw (mandibular) movement event (relevant event 714). Accordingly, based on a timestamp/time data associated with the positioning/movement data determined to be associated with the jaw movement event, other physiological data collected at the time of the timestamp/time data may be correlated and stored in association with the relevant event 714.

At OPERATION 812, results of the sleep cycle may be determined. For example, the results may include a summary of collected physiological data, which may include totals, averages, and/or extremas (e.g., maximums and/or minimums) of the collected data. For example, the summary may include a total sleep cycle time, average, minimum, and/or maximum heart rate, average, minimum, and/or maximum blood oxygen levels, etc. According to an aspect, the results may further include information about determined relevant events 714. For example, the results may include a listing of relevant events 714, and may further include additional information about the relevant events, such as measurements of the individual's airway, jaw movement measurements, occlusal force measurements, time data, graphs/visualizations 716, the data that satisfy the relevance rules, severity scores, confidence scores, comparisons, raw data, etc.

At OPERATION 814, the results may be output to one or more output devices 128. According to an aspect, the results may be provided in response to receiving an indication of a request for the results. The indication of the request may be associated with a user request, which may be received via an audible user interface, a GUI 710, etc., and the results may be provided as one or a combination of visual and audible output. The user 702 may be the individual or a healthcare professional. According to an example, the results may be used as part of diagnosing the individual with a sleep-related disorder, determining efficacy of a treatment and/or therapy, verifying effectiveness of a rendered treatment and/or therapy, educating the individual, or other clinical/educational applications.

At OPERATION 816, a dynamic 3D visualization 716c,d of a relevant event 714 may be generated. In some examples, the dynamic 3D visualization 716c,d may be generated in response to receiving an indication of a user request for the visualization. The dynamic 3D visualization may include the morph-able 3D model 722 described above, wherein the morph-able 3D model 722 may be generated based on registration of 3D image data (e.g., CBCT scan) of the individual to baseline acoustic reflection measurement data. In some examples, the morph-able 3D model 722 may be generated based on additional images (e.g., photographs, radiographs, ultrasounds, intra-oral scans) and additional baseline measurement data (e.g., baseline positioning/movement data, baseline pressure data). Portions of the 3D model 722 may be morphed to represent dynamic movements of the individual's airway and/or mandible based on received acoustic reflection measurement data, and may further include representations of dynamics of occlusal force distributions based on received pressure data.

At OPERATION 818, the dynamic 3D visualization 716c,d may be provided to an output device 128. For example, the dynamic 3D visualization 716c,d may be displayed on a display device 120, stored in removable memory 124, printed, transmitted to another computing device, etc. According to an aspect, the dynamic 3D visualization 716c,d may be provided as a video, where actual dynamic movements and anatomic changes can be played back and rendered on the display screen 120 in the GUI 710 provided by the DREAMS system 110. The method 800 may end at OPERATION 898.

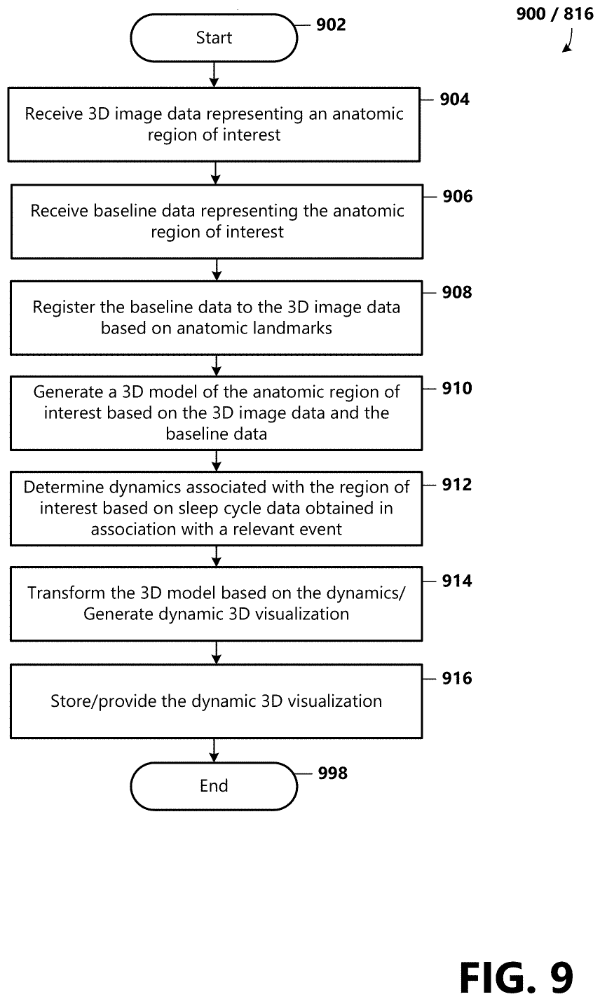

FIG. 9 is a flow diagram depicting general stages of an example process for generating a dynamic 3D visualization 716c,d of a relevant event 714. For example, the process may be used as part of OPERATION 816 in response to receiving an indication of a user request for the dynamic 3D visualization. With reference now to FIG. 9, the method 900 starts at OPERATION 902 and proceeds to OPERATION 904, where static 3D image data representing an anatomic region of interest may be received. The static 3D image data may be obtained in an offline process by a static 3D imaging device 116, such as a CBCT scanner. In some examples, the static 3D image data may be obtained while the individual is in a supine position. According to an aspect, the static 3D image data may include visualizations (e.g., sagittal, axial, coronal, and 3D views), measurements (e.g., volume and area measurements), and visual graphs of at least the individual's upper airway anatomy and mandible. According to an aspect, one or more anatomic landmarks (e.g., the tongue, oral cavity, nasal cavity, oropharyngeal junction (OPJ), oropharynx, epiglottis, hypopharynx, velopharynx, glottis, incisive papilla, hyoid bone, mental foramen, maxillary tuberosity, mandible, teeth) may be included in and may be defined from the static 3D image data. In some examples, additional image data may be received from one or more other imaging data sources 118.

At OPERATION 906, baseline data representing the anatomic region of interest may be received. For example, the baseline data may include one or more of acoustic reflection measurement data collected by the acoustic reflection measurement device 112, positioning/movement data collected by the positioning/movement sensor(s) 104, and pressure data collected by the pressure sensor(s) 106. According to an aspect, one or more anatomic landmarks (e.g., the tongue, oral cavity, nasal cavity, oropharyngeal junction (OPJ), oropharynx, epiglottis, hypopharynx, velopharynx, glottis, incisive papilla, hyoid bone, mental foramen, maxillary tuberosity, mandible, teeth) may be included in and may be defined from the baseline data. According to an aspect, the baseline data may be acquired while the individual is in a supine position. In some examples, one or more baseline readings may be acquired while the individual is performing a respiration procedure, such as a Muller's maneuver, where the airway may be collapsed responsive to, after a forced expiration, an attempt at inspiration made with a closed mouth and nose (or closed glottis).

At OPERATION 908, one or more anatomic landmarks included in the baseline data may be registered with one or more corresponding anatomic landmarks included in the static 3D image data. For example, registration of the anatomic landmarks may enable for physiological data received in association with or relative to an anatomic landmark to be mapped to the same anatomic landmark in the image data.

At OPERATION 910, a morph-able 3D model 722 representing at least the anatomic region of interest may be generated using CAD functionalities based on the static 3D image data, wherein the morph-able 3D model 722 includes registrations/mappings between the one or more anatomic landmarks included in the static 3D image data and the baseline data. In some examples, additional received image data may be superimposed on or integrated with the morph-able 3D model 722.

At OPERATION 912, anatomic dynamics associated with the relevant event 714 may be determined based on physiological data collected from one or more data sources (e.g., acoustic reflection measurement data collected by the acoustic reflection measurement device 112, positioning/movement data collected by the positioning/movement sensor(s) 104, pressure data collected by the pressure sensor(s) 106, other data collected by one or more other data source(s) 108). For example, movements, positions, forces, diameters, CSAs, etc., of portions of the individual's anatomy in relation to the one or more anatomic landmarks may be determined based on the physiological data. The dynamics may be correlated to the relevant event 714 based on time data included in the physiological data.

At OPERATION 914, the morph-able 3D model 722 may be transformed based on the determined dynamics relative to the one or more anatomic landmarks. For example, the transformation may be a time-based transformation corresponding to the physiological data readings during the relevant event 714 that represent the updated movements, positions, forces, diameters, CSAs, etc., as a function of time. Accordingly, a dynamic 3D visualization 716c,d may be generated based on the transformations.

At OPERATION 916, the dynamic 3D visualization 716c,d may be stored in the data storage 610. The dynamic 3D visualization 716c,d may further be output to one or more output devices 128. For example, the dynamic 3D visualization 716c,d may be played back and displayed on a display device 120, may be stored in removable memory 124, printed using a printing device, transmitted to another computing device 102, etc. The method 900 ends at OPERATION 998.

FIG. 10 is a block diagram illustrating physical components of an example computing device with which aspects may be practiced. The computing device 1000 may include at least one processing unit 1002 and a system memory 1004. The system memory 1004 may comprise, but is not limited to, volatile (e.g. random access memory (RAM)), non-volatile (e.g. read-only memory (ROM)), flash memory, or any combination thereof. System memory 1004 may include operating system 1006, one or more program instructions 1008, and may include sufficient computer-executable instructions for the DREAMS system 110, which when executed, perform functionalities as described herein. Operating system 1006, for example, may be suitable for controlling the operation of computing device 1000. Furthermore, aspects may be practiced in conjunction with a graphics library, other operating systems, or any other application program and is not limited to any particular application or system. This basic configuration is illustrated by those components within a dashed line 1010. Computing device 1000 may also include one or more input device(s) 1012 (keyboard, mouse, pen, touch input device, etc.) and one or more output device(s) 1014 (e.g., display, speakers, a printer, etc.).

The computing device 1000 may also include additional data storage devices (removable or non-removable) such as, for example, magnetic disks, optical disks, or tape. Such additional storage is illustrated by a removable storage 1016 and a non-removable storage 1018. Computing device 1000 may also contain a communication connection 1020 that may allow computing device 1000 to communicate with other computing devices 1022, such as over a network in a distributed computing environment, for example, an intranet or the Internet. Communication connection 1020 is one example of a communication medium, via which computer-readable transmission media (i.e., signals) may be propagated.

Programming modules may include routines, programs, components, data structures, and other types of structures that may perform particular tasks or that may implement particular abstract data types. Moreover, aspects may be practiced with other computer system configurations, including hand-held devices, multiprocessor systems, microprocessor-based or programmable user electronics, minicomputers, mainframe computers, and the like. Aspects may also be practiced in distributed computing environments where tasks are performed by remote processing devices that are linked through a communications network. In a distributed computing environment, programming modules may be located in both local and remote memory storage devices.

Furthermore, aspects may be practiced in an electrical circuit comprising discrete electronic elements, packaged or integrated electronic chips containing logic gates, a circuit using a microprocessor, or on a single chip containing electronic elements or microprocessors (e.g., a system-on-a-chip (SoC)). Aspects may also be practiced using other technologies capable of performing logical operations such as, for example, AND, OR, and NOT, including, but not limited to, mechanical, optical, fluidic, and quantum technologies. In addition, aspects may be practiced within a general purpose computer or in any other circuits or systems.

Aspects may be implemented as a computer process (method), a computing system, or as an article of manufacture, such as a computer program product or computer-readable storage medium. The computer program product may be a computer storage medium readable by a computer system and encoding a computer program of instructions for executing a computer process. Accordingly, hardware or software (including firmware, resident software, micro-code, etc.) may provide aspects discussed herein. Aspects may take the form of a computer program product on a computer-usable or computer-readable storage medium having computer-usable or computer-readable program code embodied in the medium for use by, or in connection with, an instruction execution system.

Although aspects have been described as being associated with data stored in memory and other storage mediums, data can also be stored on or read from other types of computer-readable media, such as secondary storage devices, like hard disks, floppy disks, flash drives, or a CD-ROM, or other forms of RAM or ROM. The term computer-readable storage medium refers only to devices and articles of manufacture that store data or computer-executable instructions readable by a computing device. The term computer-readable storage media does not include computer-readable transmission media.

Aspects of the present invention may be used in various distributed computing environments where tasks are performed by remote processing devices that are linked through a communications network.

Aspects of the invention may be implemented via local and remote computing and data storage systems. Such memory storage and processing units may be implemented in a computing device. Any suitable combination of hardware, software, or firmware may be used to implement the memory storage and processing unit. For example, the memory storage and processing unit may be implemented with computing device 1000 or any other computing devices 1022, in combination with computing device 1000, wherein functionality may be brought together over a network in a distributed computing environment, for example, an intranet or the Internet, to perform the functions as described herein. The systems, devices, and processors described herein are provided as examples; however, other systems, devices, and processors may comprise the aforementioned memory storage and processing unit, consistent with the described aspects.

The description and illustration of one or more aspects provided in this application are intended to provide a thorough and complete disclosure of the full scope of the subject matter to those skilled in the art and are not intended to limit or restrict the scope of the invention as claimed in any way. The aspects, examples, and details provided in this application are considered sufficient to convey possession and enable those skilled in the art to practice the best mode of the claimed invention. Descriptions of structures, resources, operations, and acts considered well-known to those skilled in the art may be brief or omitted to avoid obscuring lesser known or unique aspects of the subject matter of this application. The claimed invention should not be construed as being limited to any embodiment, aspects, example, or detail provided in this application unless expressly stated herein. Regardless of whether shown or described collectively or separately, the various features (both structural and methodological) are intended to be selectively included or omitted to produce an embodiment with a particular set of features. Further, any or all of the functions and acts shown or described may be performed in any order or concurrently. Having been provided with the description and illustration of the present application, one skilled in the art may envision variations, modifications, and alternate embodiments falling within the spirit of the broader aspects of the general inventive concept provided in this application that do not depart from the broader scope of the present disclosure.

We claim:

1. A system for providing sleep-related disorder data collection, assessment, and visual representation, the system comprising:
at least one processor;
a memory storage device including instructions that when executed by the at least one processor are configured to:
receive physiological data of an individual during a sleep cycle, wherein the physiological data at least include acoustic measurement data of the individual's airway;
analyze the physiological data for determining a relevant event associated with a sleep-related disorder;
determine results associated with the relevant event; and
provide output for display on a screen, wherein the output includes the results.

2. The system of claim 1, wherein the system is configured to determine whether measurements included in the received physiological data satisfy relevance rules associated with the relevant event.

3. The system of claim 1, wherein the system is further configured to correlate the acoustic measurement data with additional physiological data associated with the relevant event.

4. The system of claim 3, wherein the physiological data further include:
positioning and movement data of the individual's mandible; and
pressure data associated with occlusal forces.

5. The system of claim 4, wherein the physiological data further include one or more of:
blood-oxygen saturation levels;
airflow levels;
air pressure levels;
respiratory effort;
heart rate;
heart rhythm;
breathing patterns;
eye movements;
muscle activity;
brain activity; and
snoring.

6. The system of claim 1, wherein the sleep-related disorder includes one of:
apnea;
hypo-apnea;
sleep bruxism;
respiratory effort-related arousals;
obstructed airway-associated head or neck movement disorder; and
temporomandibular disorders.

7. The system of claim 6, wherein the relevant event includes one of:
jaw movement;
clenching of teeth;
airway collapse;
elevated heart rate; and
low blood oxygen saturation level.

8. The system of claim 1, wherein the output includes one or more of:
a summary of the received physiological data;
measurements of the individual's airway;
mandibular movement measurements;
occlusal force measurements;
visualizations; and
raw data.

9. The system of claim 8, wherein the visualizations include a dynamic 3D visualization of the relevant event, wherein the dynamic 3D visualization includes a morph-able 3D model that transforms in relation to at least one of:
measurements and movements of at least the individual's airway included in the physiological data; and
measurements and movements of the individual's mandible included in the physiological data.

10. A method for providing sleep-related disorder data collection, assessment, and visual representation, comprising:
receiving physiological data of an individual during a sleep cycle, wherein the physiological data at least include acoustic measurement data of the individual's airway;
analyzing the data for determining a relevant event associated with a sleep-related disorder;
determining results associated with the relevant event; and
providing output for display on a screen, wherein the output includes the results.

11. The method of claim 10, wherein determining the relevant event comprises determining whether measurements included in the received physiological data satisfy relevance rules associated with the relevant event.

12. The method of claim 10, further comprising correlating the acoustic measurement data with other physiological data associated with the relevant event.

13. The method of claim 10, wherein receiving physiological data further includes:
receiving positioning and movement data of the individual's mandible; and
receiving pressure data associated with occlusal forces.

14. The method of claim 13, further comprising generating a visualization representing the relevant event, wherein generating the visualization comprises one or more of:
generating a visualization representing measurements of the individual's airway during the relevant event;
generating a visualization representing measurements of mandibular movement during the relevant event; and
generating a visualization representing occlusal force measurements during the relevant event.

15. The method of claim 14, wherein generating the visualization representing the relevant event comprises generating a dynamic 3D visualization, wherein generating the dynamic 3D visualization comprises:
generating a morph-able 3D model based on static 3D image data and baseline data; and
transforming the morph-able 3D model in relation to measurements of the individual's airway during the relevant event.

16. The method of claim 13, further comprising one or more of:
providing audible output associated with the determined results for output via a speaker;
storing the determined results in removable memory;
printing a report including the determined results; and
transmitting the determined results to a computing device.

17. An apparatus for providing sleep-related disorder data collection, wherein the apparatus includes an oral appliance device configured to be worn by an individual during a sleep cycle, the apparatus including or operatively connected to:
an acoustic reflection measurement device configured to collect acoustic measurement data of the individual's airway;
a positioning and movement sensor configured to collect positioning and movement data associated with mandibular movements; and
a pressure sensor configured to collect pressure data associated with occlusal forces.

18. The apparatus of claim 17, wherein the acoustic reflection measurement device includes:
an acoustic wave source configured to generate piezoelectric vibrations;
an acoustic wave tube configured to:
project the piezoelectric vibrations down the individual's airway; and
allow reflected acoustic waves to travel to an acoustic reflection sensor;
the acoustic reflection sensor configured to:
capture the reflected acoustic waves; and
measure an amplitude of the reflected acoustic waves for determining measurements of the individual's airway; and
a noise control component configured to reduce external noise generated by the acoustic wave source.

19. The apparatus of claim 17, wherein the positioning and movement sensor includes:
a light source configured to emit beams of infrared or laser light onto a target surface;
an optical sensor configured to:
receive light reflected from the target surface; and
convert patterns of the reflected light into digital signals; and
a digital signal processor configured to analyze the digital signals for determining movement of the target surface relative to the optical sensor.

20. The apparatus of claim 17, wherein the pressure sensor includes a piezoelectric pressure sensing element configured to measure changes in static and dynamic occlusal forces.
